(19) United States
(12) Patent Application Publication
(10) Pub. No.: US 2023/0372425 A1
(43) Pub. Date: Nov. 23, 2023

(54) TOPICAL COMPOSITIONS CONTAINING MANUKA OIL AND PALMAROSA OIL FOR TREATING SKIN CONDITIONS

(71) Applicant: Manuka Therapeutics Ltd., Newmarket, Auckland (NZ)

(72) Inventors: Robert Gilmour, Woolloomooloo (AU); Stuart H. Cairns, Remuera (NZ); Suki Harding, Titirangi, Auckland (NZ)

(21) Appl. No.: 18/028,327

(22) PCT Filed: May 6, 2022
(86) PCT No.: PCT/IB2022/000255

§ 371 Date: Mar. 24, 2023

Related U.S. Application Data
(60) Provisional application No. 63/185,182, filed on May 6, 2021.

(51) Int. Cl. A61K 36/61; A61K 36/899; A61K 36/53; A61K 31/00; A61K 9/00; A61K 47/10; A61P 17/00; A61P 31/22

(52) U.S. Cl. CPC A61K 36/61; A61K 36/899; A61K 36/53; A61K 31/658; A61K 9/0014; A61K 47/10; A61P 17/00; A61P 31/22

(57) ABSTRACT

A topical composition for treatment of a skin condition comprising a mixture of Leptospermum scoparium oil (manuka oil or β-triketones extracted from manuka oil), at least one of Cymbopogon martinii (palmarosa oil), CBD oil or isolate, or Pogostemon cablin (patchouli oil), and a carrier composition.

### FIELD OF THE INVENTION

The application relates to topical compositions for the treatment of damaged skin in mammals, including humans.

### BACKGROUND

Various ailments, diseases, or injuries can cause skin conditions. These causes include, but are not limited to, physical injury or trauma such as lacerations, chapping, burns, blistering, and the like; diseases; physical disorders such as allergic reactions, dryness, acne, or the like; microbes including bacteria, fungi, viruses, parasites, and the like; or other causes. Damaged skin, particularly those resulting in an open wound or compromised skin barrier, may be susceptible to microbial infection, which can impede or inhibit the body's natural ability to heal. Many options and approaches have been used for treating damaged skin designed to protect or promote skin healing.

One approach for treating skin conditions as described in PCT published application WO 2021/024211 A1, which is assigned to the assignee of the present application, uses Leptospermum scoparium oil (manuka oil) and Nigella sativa seed oil (black seed oil), and a carrier composition. While this approach can produce beneficial results, the combinations described can also produce an objectionable odour that may impact its useability and acceptance.

Thus, there exists a present need in the art for improved compositions for treating damaged skin designed to protect or promote skin healing.

### SUMMARY

The present disclosure generally relates to topical compositions with antimicrobial, anti-inflammatory and/or other wound healing properties that may be useful for treating or preventing various skin infections and other ailments including, but not limited to, skin conditions that relate to impetigo, including ecthyma, skin and wound infections, dry skin conditions such as eczema, or psoriasis, and the like.

In one embodiment in accordance with this disclosure, a topical composition for treatment of a variety of skin conditions includes Leptospermum scoparium oil (manuka oil) in the form of the whole oil or components such as β-triketones and Cymbopogon martinii oil (palmarosa oil), and a carrier composition. As described further below, the combinations of manuka oil or its components (particularly β-triketones) extracted from manuka oil and palmarosa oil may provide synergistic effects that demonstrate high effectiveness against gram positive bacteria such as Staphylococcus aureus, Streptococcus pyogenes, and methicillin resistant Staphylococcus aureus (MRSA), which are recognized contributors that cause impetigo. Additionally, or alternatively, the topical compositions disclosed may be applied to damaged skin to reduce inflammation, and/or to treat and/or prevent other microbial infections and/or improve the rate of healing of the damaged area.

In another embodiment in accordance with this disclosure, a topical composition for treatment of a variety of skin conditions includes Leptospermum scoparium oil (manuka oil or its components such as β-triketones), at least one of Cymbopogon martinii (palmarosa oil), CBD or cannabidiol (e.g., natural oils containing CBD or purified CBD isolate), or Pogostemon cablin (patchouli oil), and a carrier composition. The compositions may also optionally include other oils described herein. As described further below, the combinations of manuka oil and either CBD oil or isolate, palmarosa oil, patchouli oil, or combinations thereof may provide synergistic effects that demonstrate sustained antimicrobial activity, accelerated wound healing, excellent anti-inflammatory action or all three properties, compared to other topical compositions that include the oils or actives independently. In some embodiments, the topical compositions disclosed may be applied to damaged skin to reduce inflammation and/or to treat and/or prevent microbial growth and/or improve the rate of healing of the damaged area. The compositions may also be used for the treatment or prevention of various skin conditions, such as psoriasis, or eczema, impetigo, boils, abscesses, wound infections, viral infections such as cold sores or shingles, fungal infections such as athlete's foot or yeast infections such as thrush.

In an embodiment in accordance with this disclosure, a topical composition for treatment of a skin condition may be in the form of a topical balm, topical ointment, topical gel, topical solution or topical spray. The topical composition may include a mixture of Leptospermum scoparium oil (manuka oil or its components such as β-triketones), at least one of Cymbopogon martinii oil (palmarosa oil), CBD oil or isolate, or Pogostemon cablin oil (patchouli oil), ethylhexyl palmitate and a carrier composition. In some such examples, the topical composition comprises a topical ointment that includes a mixture of Leptospermum scoparium oil (manuka oil or its components such as β-triketones) and Cymbopogon martinii oil (palmarosa oil). The manuka oil (or components such as β-triketones) and palmarosa oil may be present in a therapeutic amount, e.g., in an amount effective for the treatment of impetigo.

In another embodiment in accordance with this disclosure, a topical composition for treatment or prevention of a skin condition such as eczema or psoriasis may be in the form of a topical hydrophilic ointment or cream. The topical composition including a mixture of Leptospermum scoparium oil (manuka oil or components such as β-triketones extracted from manuka oil), at least one of CBD oil, Cymbopogon martinii oil (palmarosa oil), or Pogostemon cablin oil (patchouli oil), and a carrier composition containing ingredients that may include polyethylene glycol (PEG) or macrogol, allantoin, aloe, disodium EDTA, phenoxyethanol, decadiene cross polymer, triethylene glycol, stearyl alcohol, white soft paraffin, propylene glycol, lactic acid, glycerol and water.

In another embodiment in accordance with this disclosure, a topical composition for treatment of a skin condition such as impetigo comprises a mixture of Leptospermum scoparium oil (manuka oil or components such as β-triketones extracted from manuka oil), Cymbopogon martinii oil (palmarosa oil), and a carrier composition. The manuka oil (or components such as β-triketones) and palmarosa oil may be present in a therapeutic amount, e.g., in an amount effective for the treatment of impetigo. In some such examples, the composition may include a ratio of palmarosa oil to manuka oil or β-triketones of about 3:1 to about 1:3, about 2:1 to about 1:2, or about 1:1. The carrier composition may contain polyethylene glycol (PEG) or natural or naturally derived ingredients.

In another embodiment in accordance with this disclosure, an antimicrobial topical composition for treatment of a skin condition such as microbial skin infections such as impetigo, boils, sties, abscesses, folliculitis, cellulitis, carbuncles, furuncles, wound infections, viral infections, fungal infections and yeast infections is provided. The topical composition comprises a mixture of Leptospermum scoparium oil (manuka oil or its components such as β-triketones), and one or more of Cymbopogon martinii oil (palmarosa oil), CBD oil or isolate, Pogostemon cablin oil (patchouli oil), or combinations thereof, and a carrier composition.

In another embodiment in accordance with this disclosure, a method for treating or preventing a skin condition comprising applying a topical composition to an area containing a skin condition, the topical composition comprising a mixture of Leptospermum scoparium oil (manuka oil) or its components (for example β-triketones), and one of more of Cymbopogon martinii oil (palmarosa oil), CBD (oil or isolate), Pogostemon cablin oil (patchouli oil), or combinations thereof, and a carrier composition.

In another embodiment in accordance with this disclosure, a method for manufacturing a topical composition used to treat a skin condition comprising mixing the topical composition comprising a mixture of Leptospermum scoparium oil (manuka oil) or its components (for example β-triketones), and one or more of Cymbopogon martinii oil (palmarosa oil), CBD oil or isolate, Pogostemon cablin oil (patchouli oil), or combinations thereof, and other ingredients into a carrier composition.

In another embodiment, the disclosed topical composition may include a mixture of Leptospermum scoparium oil (manuka oil) or its components (such as β-triketones) and one or more of Nigella sativa seed oil (black seed oil), Matricaria recutita oil (German chamomile), α-bisabolol, Elettaria cardamomum oil (cardamom), Thymus vulgaris oil (thyme), Origanum vulgare oil (oregano), Santalum album oil (sandalwood), Illicium verum oil (star anise), Mentha spicata oil (spearmint), Centella asiatica (gotukola extract), β-caryophyllene, Psoralea corylifolia oil (babchi or bakuchi), Terminalia ferdinandiana oil (Kakadu plum), or combinations thereof. Additionally or alternatively, the disclosed topical compositions may include a mixture of Leptospermum scoparium oil (manuka oil or its components such as β-triketones), Cymbopogon martinii oil (palmarosa oil), CBD (oil or isolate), Pogostemon cablin oil (patchouli oil), or a combination thereof, a carrier composition, and one or more of: Nigella sativa seed oil (black seed oil), Matricaria recutita oil (German chamomile), α-bisabolol, Elettaria cardamomum oil (cardamom), Thymus vulgaris oil (thyme), Origanum vulgare oil (oregano), Santalum album oil (sandalwood), Illicium verum oil (star anise), Mentha spicata oil (spearmint), Centella asiatica (gotukola extract), β-caryophyllene, Psoralea corylifolia oil (babchi or bakuchi), Terminalia ferdinandiana oil (Kakadu plum), or combinations thereof.

The above summary is not intended to describe each disclosed embodiment, or every implementation of the embodiments disclosed herein. The description that follows more particularly exemplifies illustrative examples. In several places throughout the application, guidance is provided through lists of examples, which can be used in various combinations. In each instance, the recited list serves only as a representative group and should not be interpreted as an exclusive list.

### BRIEF DESCRIPTION OF THE DRAWINGS

FIG. 1 is a table demonstrating antimicrobial data for ECMT-154 PEG ointment and references, according to embodiments of the present disclosure.

FIG. 2 is a table of demonstrating the effect of ECMT-154 topical compositions and references against C. acnes and S. epidermidis, according to embodiments of the present disclosure.

FIG. 3 is a table demonstrating the wound healing effect of manuka oil β-triketones and palmarosa oil, according to embodiments of the present disclosure.

FIG. 4A is a graphs showing skin barrier repair study results for 1 hour treatment of skin with 0.2% MBS β-triketones+0.2% palmarosa oil, according to embodiments of the present disclosure.

FIG. 4B is graphs showing 1 hour treatment of skin with 0.2% MBS β-triketones+0.2% patchouli oil, according to embodiments of the present disclosure.

FIG. 5 is a graph showing GCMS results for manuka oil β-triketones with a table summarizing the results, according to embodiments of the present disclosure.

FIG. 6 is a graph of GCMS results for manuka oil β-triketones, palmarosa oil and the combined composition, according to embodiments of the present disclosure.

FIG. 7 is tables showing HRIPT results for a topical composition comprising manuka oil β-triketones, palmarosa oil in a polyethylene glycol carrier base, according to embodiments of the present disclosure.

### DEFINITIONS

As used herein, “a,” “an,” “the,” “at least one,” and “one or more” are used interchangeably. Thus, for example, a composition that comprises “a” surfactant can be interpreted to mean that the composition includes “one or more” surfactants.

As used herein, the term “CBD oil” is used to refer to oils (e.g., natural plant oils) containing cannabidiol (CBD). CBD oils man include essential oil extracts from plants of genus Cannabis (e.g., Cannabis oil, hemp oil, or oils from the Cannabaceae family). Such oils may be processed to remove certain cannabinoids from the oils or to increase the concentration, consistency or purity of CBD, e.g., CBD isolate. Additionally, or alternatively, CBD oils may refer to other oil products such as natural plant-based oils containing CBD. Such oils may include coconut oil, hemp seed oil, jojoba oil, rosehip oil, or other oil that act as a carrier for CBD.

As used herein, the term “consists substantially of” means that at least 95 percent by weight (wt. %) the respective material is made of the recited component. For example, a carrier composition that consists substantially of natural or vegan materials means that at least 95 wt. % of the composition is made of natural or vegan materials.

As used herein, the term “consists essentially of” means that the respective material is formulated of the recited components, but also may include other contaminants that do not materially affect the physical or chemical properties of the material. In some examples, a composition that “consists essentially of” a list of materials may include less than 0.5 wt. % of other components.

As used herein, the term “comprises” and variations thereof do not have a limiting meaning where these terms appear in the description and claims.

As used herein, the term “essential oil,” “plant oil,” or “seed oil,” refers to a natural, plant-based oil. Unless indicated otherwise, such oils may be derived from steam distillation, a cold press process or CO₂ extraction. The method of producing such oils (e.g., steam distillation versus cold press) may alter the compositional makeup and physical and medicinal properties of the oil.

As used herein, the term “excipient” refers to an ingredient or component of a mixture or composition. Unless otherwise indicated, an excipient may be an active, inactive, or inert component or ingredient of a mixture, according to embodiments of the present disclosure.

As used herein, the terms “mixture” or “mixing” refers to a physical mixture or physically mixing (e.g., blending) two or more components together. Unless otherwise indicated, a mixture includes emulsions, heterogeneous mixtures, and homogeneous mixtures.

As used herein, the term “natural” describes a bioactive ingredient such as an essential oil, essential oil constituent or an excipient derived from a renewable source such as plants, insects, or animal-based sources, as opposed to synthetic- or petroleum-based sources. For example, the phrases “natural oil”, “natural wax” or “natural extract” may include oils, waxes, extracts and other ingredients derived from plants, insects (e.g., bees), or other animals. A bioactive ingredient or an excipient may still be considered “natural” even though the excipient may have undergone a chemical or physical extraction process. For example, essential oils are typically extracted from plants through a distillation process. Essential oils are still considered natural even though processing has occurred to isolate and purify the oil.

As used herein, the terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

As used herein, the term “topical composition” refers to compositions designed to be applied to the skin of a patient and is not intended for internal consumption or use. Such compositions may include delivery systems (e.g., liposomal encapsulation systems or other nanomaterials), transdermal excipients (e.g., those that penetrate or pass through the layers of the epidermis) and may in some examples, be applied to portions of the skin that include a skin condition or wound (e.g., infection, laceration, abrasion, rash, dryness, diagnosed skin condition, inflammation, surgical incision, and the like). Topical compositions may include, but are not limited to balms, creams, gels, lotions, ointments, solutions, sprays, oils or the like. Topical compositions may be applied directly to the skin of a patient (e.g., direct application of a balm comprising the composition) or indirectly to the skin of a patient (e.g., application of a wound dressing comprising the composition that is applied to the skin).

Also herein, the recitations of numerical ranges by endpoints include all numbers subsumed within that range (e.g., 1 to 5 includes 1, 1.5, 2, 2.75, 3, 3.80, 4, 5, etc.). Furthermore, disclosure of a range includes disclosure of all sub-ranges included within the broader range (e.g., 1 to 5 discloses 1 to 4, 1.5 to 4.5, 4 to 5, etc.).

### DETAILED DESCRIPTION

The embodiments described in this disclosure provide a topical composition for the treatment of various skin conditions (e.g., impetigo, eczema, psoriasis, inflammatory conditions, boils, sties, abscesses, folliculitis, cellulitis, carbuncles, furuncles, wound infections, viral infections, fungal infections and yeast infections) and method of producing and using such compositions. Various embodiments are effective at treating many forms of skin conditions including damage due to abrasions, disease, exposure to toxins and radiation, immune responses, injury, medical procedures, microbes such as bacteria, viruses, fungi, yeasts and parasites, trauma, and the like.

In some embodiments, the disclosed topical compositions may be formulated to provide therapeutic treatment of the bacterial skin condition impetigo. Impetigo is a common, highly contagious skin infection and mainly affects infants and children. Impetigo, also referred to as “school sores”, usually appears as red sores on a person's face, especially around the person's nose and mouth, and on hands and feet. Classic signs and symptoms of impetigo involve red fluid filled sores that can rupture or weep and form honey-colored crust over the sore. While the sores typically occur around the nose and mouth, the sores can be spread to other areas of the body through contact transfer such as by fingers, clothing, towels, and the like.

A less common form of the impetigo disorder, called bullous impetigo, may feature larger blisters that occur on the buttocks of infants and young children. Another more serious form of impetigo, called ecthyma, penetrates deeper into skin tissue, and produces painful fluid-filled sores that can develop into ulcers.

The disclosed topical compositions may also be used in treating other skin conditions. While in general, the compositions are described in the context of treating human skin conditions, the compositions may also be used, where applicable, in treating skin conditions of other mammals, including those with fur, such as in practices of veterinary medicine. Skin pathologies frequently have both an infective and an inflammatory component. The infective component is often a mixed infection rather than purely bacterial or purely viral, although there are well known exceptions, but a composition which has strong overall antimicrobial data and strong anti-inflammatory data may provide a product with broad efficacy. As disclosed herein, combining natural bioactives can provide a product with optimized efficacy in both components.

The disclosed topical compositions include a unique mixture of Leptospermum scoparium oil (manuka oil or its components, particularly β-triketones), at least one of Cymbopogon martinii (palmarosa oil), CBD (oil or isolate), or Pogostemon cablin Oil (patchouli oil), and a carrier composition. More specifically, the topical compositions may include at least manuka oil or its components (particularly β-triketones) and palmarosa oil. The compositions may also optionally include Nigella sativa seed oil (black seed oil), Matricaria recutita oil (German chamomile), α-bisabolol, Elettaria cardamomum oil (cardamom), Thymus vulgaris oil (thyme), Origanum vulgare oil (oregano), Santalum album oil (sandalwood), Illicium verum oil (star anise), Mentha spicata oil (spearmint), Centella asiatica (gotukola extract), β-caryophyllene, Psoralea corylifolia oil (babchi or bakuchi), or Terminalia ferdinandiana oil (Kakadu plum), and ethylhexyl palmitate, or a combination of these ingredients.

As discussed further below, the inclusion of manuka oil or its components, particularly β-triketones, in combination with palmarosa oil, CBD oil or isolate, patchouli oil, or combinations thereof may provide a synergistic effect when treating various topical skin conditions such as impetigo, eczema, psoriasis, fungal or viral skin infections or acne. In some cases, synergistic effects may also arise when manuka oil or its components (particularly β-triketones) are used in combination with Nigella sativa seed oil (black seed oil), Matricaria recutita oil (German chamomile), Elettaria cardamomum oil (cardamom), Thymus vulgaris oil (thyme), Origanum vulgare oil (oregano), Santalum album oil (sandalwood), Illicium verum oil (star anise), Mentha spicata oil (spearmint), Centella asiatica (gotukola extract), β-caryophyllene, Psoralea corylifolia oil (babchi or bakuchi), or Terminalia ferdinandiana oil (Kakadu plum).

Additionally, when treating various topical skin infections such as bacterial infections, e.g., impetigo and boils, viral infections such as cold sores and shingles, fungal infections such as athlete's foot, yeast infections such as thrush, and wound infections, the inclusion of at least one of palmarosa oil, patchouli oil or CBD oil in the disclosed manuka oil (or manuka oil β-triketone)-based compositions may provide further synergistic effects that increase the antimicrobial activity and/or duration of effectiveness of the topical composition as compared to a topical composition comprising only manuka oil. In some cases, additional synergistic antimicrobial activity may be achieved when Nigella sativa seed oil (black seed oil), Matricaria recutita oil (German chamomile), Elettaria cardamomum oil (cardamom), Thymus vulgaris oil (thyme), Origanum vulgare oil (oregano), Santalum album oil (sandalwood), Illicium verum oil (star anise), Mentha spicata oil (spearmint), Centella asiatica (gotukola extract), β-caryophyllene, Psoralea corylifolia oil (babchi or bakuchi), or Terminalia ferdinandiana oil (Kakadu plum) are included in the manuka oil (or manuka oil β-triketone)-based composition. The use of nano-delivery systems such as liposomes may further enhance these effects.

As demonstrated below, manuka oil β-triketones and palmarosa oil have proven exceptionally beneficial and effective toward the treatment of impetigo. Palmarosa oil or patchouli oil may be particularly beneficial when combined with manuka oil (or manuka oil β-triketone)-based compositions for the treatment of inflammatory skin conditions such as eczema or psoriasis. The therapeutic combination may be further enhanced when formulated as an ointment or cream that includes at least one of polyethylene glycol (e.g., PEG 300, PEG 3350, and the like), allantoin, aloe, disodium EDTA, phenoxyethanol, decadiene cross polymer, triethylene glycol, stearyl alcohol, white soft paraffin, propylene glycol, lactic acid, glycerol, and water.

Additionally or alternatively, the disclosed topical compositions may include ethylhexyl palmitate, which can also enhance the physical properties of the topical compositions, e.g., topical balms, and help retain the disclosed oils within the composition against the user's skin while simultaneously promoting hydration and skin healing conditions. The topical compositions disclosed may also provide a protective barrier that enhances healing for a variety of skin conditions as well as enhance the tactile properties and wearability attributes of the topical composition.

In accordance with various embodiments, the disclosed topical compositions include Leptospermum scoparium oil (manuka oil) or its components (particularly β-triketones). Manuka oil may be extracted from the leaves of a Leptospermum scoparium plant using, for example, a steam distillation process. Leptospermum scoparium is the botanical name given to New Zealand's Manuka tree. There are two subordinate taxa of Leptospermum scoparium in New Zealand, namely Leptospermum scoparium var. incanum, which typically has pinkish red-centered flowers and Leptospermum scoparium var. scoparium, which typically has white flowers. While the Manuka tree is sometimes referred to as the New Zealand Tea Tree, oil extracted from the Manuka tree should not be confused with “tea tree oil”. In contrast to manuka oil, conventional tea tree oil is derived from the plant Melaleuca alternfolia, commonly found in Australia. Both the Melaleuca alternfolia and Leptospermum scoparium belong to the myrtle family Myrtaceae commonly known as tea trees however, the oils extracted from each have comparatively different properties and entirely different chemical compositions.

Manuka oil includes three major groups of compounds including monoterpenes, sesquiterpenes, and β-triketones. Monoterpenes are present at low levels (typically less than 5%) and can include, for example, α- and β-pinene and myrcene. Sesquiterpenes are predominant (typically more than 60%) and can include, for example, trans-calamenene, δ-cadinene, cadina-1,4-diene, α-copaene α- and β-selinene, aromadendrene, and β-caryophyllene. β-triketones include, for example, flavesone, iso-leptospermone, leptospermone, and grandiflorone. Without being bound to a specific theory, the β-triketones are believed to contribute to the exceptional antimicrobial properties of manuka oil and a synergistic effect of these properties is logarithmically enhanced when manuka oil with high β-triketones is combined with a composition containing palmarosa oil, and/or patchouli oil, and/or CBD (oil or isolate). In some embodiments, this synergistic effect may be further enhanced in a combination with other natural bioactives such as Nigella sativa seed oil (black seed oil), Matricaria recutita oil (German chamomile), Elettaria cardamomum oil (cardamom), Thymus vulgaris oil (thyme), Origanum vulgare oil (oregano), Santalum album oil (sandalwood), Illicium verum oil (star anise), Mentha spicata oil (spearmint), Centella asiatica (gotukola extract), β-caryophyllene, Psoralea corylifolia oil (babchi or bakuchi), or Terminalia ferdinandiana oil (Kakadu plum).

The composition of manuka oil (Leptospermum scoparium) is dependent on its chemotype of chemical identity. More than ten chemotypes of New Zealand manuka have been identified. The amount of β-triketones contained within manuka oil extracted from manuka leaves depends on the region within New Zealand in which the Manuka tree is harvested. For example, Manuka trees harvested from the East Cape region of New Zealand are classified as high triketone chemotype, and contain the highest levels of β-triketones compared to any other region, presumably due to genetic, epigenetic and environmental factors such as isolated position of the East Cape region, temperature, humidity, rainfall, luminosity, UV radiation levels, altitude, soil and nutrient conditions, seasonality, circadian cycles and such like. The levels of β-triketones in manuka leaf oil extracted from Manuka trees grown the East Cape region of New Zealand can be greater than 5 weight percent (wt. %), often greater than 20 wt. %, and in some examples greater than 30 wt. %, as compared to Manuka trees harvested from other regions of New Zealand, which are typically far less than 15 wt. %, and most commonly less than 5 wt. % β-triketones. In some examples, the manuka oil used in the disclosed topical compositions may be derived from Manuka trees sourced from the East Cape region of New Zealand that possess a concentration of β-triketones of greater than 5 wt. %, greater than 10 wt. %, or greater than 20 wt. %. The high therapeutic effect of East Cape manuka oil have been attributed to the high β-triketone levels in these oils.

In preferred embodiments, the topical compositions disclosed include manuka oil having a relatively high concentration of β-triketones. In some examples, the manuka oil may include at least 10 wt. % β-triketones, at least 20 wt. % β-triketones, at least 30 wt. % β-triketones, and most preferably at least 40 wt. % β-triketones. In some examples, a sequential steam distillation process may be used to extract the pure β-triketones that are used in the disclosed topical compositions. Sourcing the manuka oil from manuka trees harvested from the East Cape region of New Zealand provides a high concentration of β-triketones in the extracted oil, a unique combination of such β-triketone materials, and likewise preserves the other components present in the extracted oil. Such unique combinations are responsible for the superior therapeutic effects of the East Cape sourced manuka oil.

In some examples, the amount of manuka oil included in a topical composition may be characterized based on the total weight of β-triketones present in the topical composition. In order to obtain sufficient amount of β-triketones in the final formulation without including excessive amounts of manuka oil in the topical composition that may otherwise diminish the physical properties and therapeutic values of the composition, the manuka oil may advantageously be sourced from the East Cape region of New Zealand to ensure a high base β-triketone content.

Different grades of manuka oil with different levels of β-triketones are commercially available. including, for example, MDTK™ 20+. MDTK™ 20+ indicates that the manuka oil with this rating is β-triketone-rich, with at least 20% β-triketones. Manuka Bioscience characterizes its MPTK™ 20+ and higher grades as being sourced from the Manuka trees harvested from the East Cape region of New Zealand. See: https://manukabioscience.co.nz and https://manukabiologicals.co.nz/products/manuka-oil-mbtk20/

In certain embodiments, the topical composition comprises about 0.05 weight percent (wt. %) to about 10 wt. % manuka oil, or β-triketones which may be extracted from manuka oil, and about 0.05 wt. % to about 10 wt. % palmarosa oil based on a total weight of the topical composition. In certain embodiments, the topical composition comprises about 0.05 weight percent (wt. %) to about 2 wt. % manuka oil, or β-triketones which may be extracted from manuka oil, and about 0.05 wt. % to about 2 wt. % palmarosa oil based on a total weight of the topical composition. In certain embodiments, the topical composition comprises about 0.05 weight percent (wt. %) to about 1 wt. % manuka oil, or β-triketones which may be extracted from manuka oil, and about 0.05 wt. % to about 1 wt. % palmarosa oil based on a total weight of the topical composition.

As discussed further below, manuka oil and β-triketones from manuka oil have high antimicrobial activity. In some examples, topical compositions disclosed that include whole manuka oil or β-triketones from manuka oil may be useful in preventing or inhibiting microbial growth associated with skin abrasions, lacerations, acne, bacterial infections, fungal infections, yeast infections, viral infections, and the like. Manuka oil and β-triketones from manuka oil exhibit good antimicrobial activity against a variety of microbes (e.g., bacteria, fungi, viruses, and the like) including, but not limited to, gram positive bacteria such as Staphylococcus aureus, MRSA, Streptococcus pyogenes, Staphylococcus epidermidis, Cutibacterium acnes, Bacillus subtilis, Enterococcus faecium, Enterococcus faecalis and Corynebacterium species, fungi and yeasts such as Candida albicans, Candida glabrata, Trichophyton mentagrophytes and Trichophyton rubrum, viruses such as Herpes simplex 1 and 2, Herpes zoster and others. Manuka oil can also be effective against gram negative bacteria such as Aggregatibacter actinomycetemcomitans, Porphyromonas gingivalis, Fusobacterium nucleatum, Shigella flexneri and Shigella sonnei.

The disclosed compositions may also include Cymbopogon martinii oil (palmarosa oil). Cymbopogon martinii refers to a species of grass in the genus Cymbopogon (lemongrasses) that is native to India, Nepal, and Indochina. The essential oil of palmarosa may be extracted from the grass leaves and inflorescence using a steam distillation process. Additional common names of palmarosa oil include Indian geranium, palm rose, rosha, and rosha grass. Palmarosa oil has at least two chemotypes: Motia and Sofia. One of the primary components of palmarosa oil is geraniol. The Motia chemotype may be considered superior for therapeutic purposes since it has a higher concentration of geraniol (around 85-92%) compared to Sofia (around 60-70%). In some examples, the palmarosa oil used in the disclosed topical compositions may be of the Motia chemotype.

Palmarosa oil may be useful in treating certain skin conditions when used in combination with manuka oil or β-triketones from manuka oil. For example, the combination of palmarosa oil and β-triketones from manuka oil has been observed to enhance the antimicrobial, anti-inflammatory, and wound healing properties of the resultant topical composition compared to the properties that may be obtained by using either oil individually. The combination may be particularly suited for treating topical skin conditions such as impetigo, boils, sties, abscesses, wound infections, viral infections, fungal infections, yeast infections eczema, psoriasis, acne, or the like. The disclosed compositions may exhibit higher efficacy against bacterial, fungal, and yeast growth, promotion of wound healing properties, lower minimum inhibitory concentrations (MIC) and minimum bactericidal concentrations (MBC), improve the efficacy or longevity of antimicrobial or anti-inflammatory properties, and the like.

In particular, topical compositions that include manuka oil or β-triketones from manuka oil and palmarosa oil may be particularly suited for treating impetigo. Geraniol in palmarosa oil contributes to the antimicrobial, anti-inflammatory and wound healing properties of palmarosa oil. Palmarosa oil may be used on lacerated or damaged skin to help prevent or inhibit bacterial growth and thereby promote skin healing. The anti-inflammatory properties of palmarosa oil may also help to soothe irritation and reduce swelling or redness of inflamed skin.

The combination of palmarosa oil and manuka oil or manuka oil β-triketones produce several surprising synergistic effects when mixed together in a topical composition. For example, the mixture of palmarosa oil and manuka oil β-triketones increases the antimicrobial activity of the composition compared to either of the two oils individually. Additionally, the mixture of palmarosa oil and manuka oil β-triketones shows a broader spectrum of antimicrobial activity and improved activity for longer periods of time than either alone.

Minimum Inhibitory Concentration (MIC) testing has demonstrated that the combination of manuka oil or β-triketones extracted from manuka oil and palmarosa oil are highly effective against gram positive bacteria such as Staphylococcus aureus, Streptococcus pyogenes, and methicillin resistant Staphylococcus aureus (MRSA) that cause impetigo (school sores) and other bacterial infections. The mode of antibacterial action of manuka oil involves the disruption of the bacterial cell membrane and leakage of cell contents.

The complexity and diversity of the various components contained within both oil extracts may play a key role in this process, and make it difficult for mutations of such microbes to develop resistance. In some examples, the topical compositions may include a ratio of palmarosa oil to manuka oil of about 3:1 to about 1:3, more preferably about 2:1 to about 1:2 or about 1:1, based on the weight percentage of β-triketones to palmarosa oil in the topical composition. The total β-triketone content may be calculated based on the supplier specifications. In other examples, β-triketones extracted from manuka oil may be used in place of the whole oil. The total amount of palmarosa oil in the topical compositions may be any suitable therapeutic amount and may be dependent on the type of topical composition being produced. In some examples, the topical compositions may include about 0.1 wt. % to about 10 wt. % palmarosa oil, more preferably about 1 wt. % to about 3 wt. %, or about 2 wt. %.

In other examples, the disclosed compositions may include manuka oil (or β-triketones extracted from manuka oil) and CBD oil as defined above includes oils, preferably natural oil, that include cannabidiol (CBD) or purified isolate form of CBD. CBD may be extracted from plants of the genus Cannabis. There are three primary species of plants recognized as being part of the Cannabis genus used in the production of CBD which include Cannabis sativa, Cannabis indica, and Cannabis ruderalis although additional species are being considered. More than 100 cannabinoids compounds may be extracted from cannabis plants with Cannabidiol (CBD) and tetrahydrocannabinol (THC) being the two primary extracts. THC is the main psychoactive element in cannabis plants and may be strictly regulated in several jurisdictions. CBD, however, is a non-psychoactive element and has gained interest for its medicinal properties. While CBD is of primary interest, the vast array of over 100 cannabinoids known to exist in cannabis plants can also contribute to the medicinal effects of CBD oil or cannabis-based oils.

CBD is also a major constituent in hemp and hemp extracts. As described above, CBD oils refer to oil-based carriers (e.g., cannabis oil, hemp oil, coconut oil, jojoba oil, rosehip oil and the like) that include CBD. For example, CBD oil may include only CBD as the active ingredient (excluding tetrahydrocannabinol [THC] or terpenes) or may include CBD-dominant oils (e.g., those where CBD is the predominate cannabinoid) and may include other cannabinoids therein. In some examples, the CBD oil may be a CBD isolate having a minimum CBD content of about 99 wt. %. In some examples, the disclosed CBD oil may have a THC content of less the limits permitted by local regulations, less than about 0.2 wt. %, or may be substantially free (e.g., free or nearly free) of THC.

CBD oil may be useful in treating certain skin conditions, particularly when used in combination with manuka oil. For example, the combination of manuka oil (or β-triketones from manuka oil) and CBD oil has been observed to enhance the anti-inflammatory and antimicrobial properties of the resultant topical composition compared to the properties that may be obtained by using either oil individually. The combination may be particularly suited for treating topical skin conditions such as eczema, psoriasis, inflammation, acne or the like.

Additionally, or alternatively, the topical compositions may include Pogostemon cablin oil (patchouli oil), which is a species of plant within the Lamiaceae family. The oil may be extracted from leaves of the Pogostemon cablin plant, which are typically native to the tropical regions of Asia. The essential oil may be extracted from the leaves of the plant using a steam distillation process or other appropriate technique. The main chemical component of patchouli oil is patchoulol, a sesquiterpene alcohol.

Patchouli oil may be useful in treating certain skin conditions including, but not limited to, acne, dermatitis, eczema, psoriasis, dry or cracked skin, dandruff, and fungal or bacterial conditions. Combinations of manuka oil (or β-triketones from manuka oil) and patchouli oil may act synergistically in n treating one or more of the above conditions. For example, the combination has been observed to enhance the anti-inflammatory and healing properties of the resultant topical composition compared to the properties that may be obtained by using either oil individually. The combination may be particularly suited for treating topical skin conditions such as dry and cracked skin, eczema, psoriasis or the like. The disclosed compositions may exhibit higher efficacy against bacterial and yeast growth, promotion of wound healing properties, lower MIC, improve the efficacy or longevity of antimicrobial properties, and the like than compositions comprising either active component individually.

The disclosed topical compositions may include at least one of, at least two of, or all three of palmarosa oil, CBD oil, or patchouli oil. In some examples, the disclosed topical compositions may include palmarosa oil, CBD oil, or patchouli oil in combination with the manuka oil (or β-triketones from manuka oil) to further enhance one or more of the characteristics of the formulation. Optionally Nigella sativa seed oil (black seed oil), Matricaria recutita oil (German chamomile), α-bisabolol, Elettaria cardamomum oil (cardamom), Thymus vulgaris oil (thyme), Origanum vulgare oil (oregano), Santalum album oil (sandalwood), Illicium verum oil (star anise), Mentha spicata oil (spearmint), Centella asiatica (gotukola extract), β-caryophyllene, Psoralea corylifolia oil (babchi or bakuchi), or Terminalia ferdinandiana oil (Kakadu plum) may also be included in the formulation.

The disclosed compositions may include, if present, about 0.1 wt. % to about 10 wt. % palmarosa oil, if present, about 0.01 wt. % to about 10 wt. % CBD oil, and if present, about 0.05 wt. % to about 10 wt. % patchouli oil.

In some examples, the topical compositions may include a carrier containing one of more of polyethylene glycol (PEG), allantoin, aloe, disodium EDTA, phenoxyethanol, decadiene cross polymer, triethylene glycol, stearyl alcohol, white soft paraffin, propylene glycol, lactic acid, glycerol and water. As indicated below, PEG has been particularly useful at enhancing the therapeutic efficacy of topical ointments that include manuka oil (or β-triketones extracted from manuka oil) and palmarosa oil. PEGs are petroleum-based compounds that are widely used as pharmaceutical excipients and in oral, topical and intravenous therapeutic formulations. It is also used in the cosmetic industry as a humectant, permeation enhancer, skin conditioner, softener, solvent, emulsifier, thickener or as part of a base carrier.

In some examples, the topical compositions disclosed, particularly topical balms, may include ethylhexyl palmitate. Ethylhexyl palmitate is a natural, organic alternative to silicone-based materials and acts as a non-occlusive emollient that provides a dry-slip, silky feel reminiscent of silicone-based products. As a non-occlusive emollient, the ethylhexyl palmitate does not inhibit the passage of air and oxygen through the topical composition to promote skin-healing conditions. The ethylhexyl palmitate may be particularly useful for skin conditions that relate to dry skin such as, for example, eczema, psoriasis, and other inflammatory skin conditions. The ethylhexyl palmitate may keep the skin moist and supple by reducing water loss from the epidermis, which in turn can promote skin healing. Additionally, ethylhexyl palmitate may act as a lubricant within the topical composition to help reduce friction between the skin and any external contact to prevent chafing and promote healing. In some formulations, the ethylhexyl palmitate may also function as a solvent to help dissolve some of the other ingredients to allow for a more homogeneous mixture.

The ethylhexyl palmitate formulated with the disclosed oils may improve the long-term effectiveness of the formulation. For example, ethylhexyl palmitate is believed to reduce the volatility of the oils in the topical composition (e.g., manuka oil, CBD oil, palmarosa oil, patchouli oil, and the like) thereby enhancing the retention of the oils within the topical composition for an extended period of time while also promoting an optimized environment for skin healing. As a result, the topical composition may include lower levels of one or more of the disclosed oils compared to other formulations while still maintaining sufficient levels of the oils to obtain the disclosed anti-inflammatory or antimicrobial properties. The ethylhexyl palmitate may also allow the topical composition to have a longer storage shelf life.

In some examples, the topical compositions disclosed may include any suitable ratio or combination of the excipients disclosed above. The topical composition may include about 0.05 wt. % to about 10 wt. % of manuka oil (or β-triketones extracted from manuka oil); about 0.01 wt. % to about 10 wt. % CBD oil (e.g., about 0.05-10 wt. %), about 0.1 wt. % to about 10 wt. % palmarosa oil (e.g., about 0.2-10 wt. %), about 0.05 wt. % to about 10 wt. % patchouli oil, or combinations thereof; and about 1 wt. % ethylhexyl palmitate based on the total weight of the topical composition with the balance provided by the carrier composition. The amount of materials may be more or less to obtain the desired characteristics depending on the carrier material used.

In addition to the Leptospermum scoparium oil (manuka oil or β-triketones extracted from manuka oil) and at least one of CBD oil, Cymbopogon martinii (palmarosa oil), or Pogostemon cablin oil (patchouli oil), the disclosed topical compositions may also include one or more ingredients selected from black seed oil, sandalwood oil, thyme oil, oregano oil, cardamom oil, spearmint oil, German chamomile oil, α-bisabolol, β-caryophyllene, star anise oil, babchi or bakuchi oil, kanuka oil, Kakadu plum seed oil, or gotukola extract.

Any suitable carrier may be added to the disclosed compositions. In some examples, the carrier composition may include natural or vegan materials. For example, among modern sophisticated consumers, there exists a strong desire and preference to use natural, environmentally friendly materials rather than synthetic- or petroleum-based materials. Accordingly, in preferred embodiments, the carrier composition may consist substantially of or may consist essentially of natural or vegan based materials. Likewise, it may be desirable for the topical composition to consist substantially of or consist essentially of natural or vegan based materials.

The topical compositions may be formulated into any suitable type of vehicle including, for example, balms, creams, gels, lotions, ointments, solutions, sprays, oils or the like. In preferred embodiments, the topical composition is formulated into a topical cream, balm or gel. Topical balms are composed of predominately non-volatile materials such that the balm remains as a solid film when applied to the skin. In contrast, ointments, creams, or gels are predominantly composed of a volatile carrier (e.g., water or alcohol) that may evaporate upon application. In some examples, the carrier composition may include water as a major component (e.g., more than 25% of the composition is water). Water-based compositions may be particularly useful in the formation of a topical gel, cream, or ointment, allowing for a portion of the composition to evaporate after application.

The disclosed oils may be incorporated into dermal delivery systems such as liposomes, niosomes, transfersomes, lipid nanoparticles, polymeric microparticles, nanoparticles, nicrofibres, or nanofibres to enhance the penetration of therapeutic bioactive ingredients in the topical compositions into the skin.

Representative topical ointments or creams that include the disclosed oils are discussed herein. However, other topical compositions that are not described herein may also be used, provided they include the disclosed manuka oil (or β-triketones from manuka oil) and palmarosa, CBD, or patchouli oil-based compositions described herein. In some embodiments, the carrier composition may include an ointment- or cream-based carrier. Such carriers may be including commercially available prefabricated ointment- or cream-based carriers that allows the disclosed oils to be mixed therein. Such carriers may include water based (e.g., water, aloe or the like) or fatty alcohol based materials (e.g., polyethylene glycol). In some examples, the carrier may be composed of predominately of water, one or more alcohols, or a combination thereof. Suitable alcohols may include, but are not limited to a combination of polyethylene glycols (e.g., PEG 4000, PEG 3350, PEG 600, PEG 400 or PEG 300), stearyl alcohol, propylene glycol, triethylene glycol, or the like. Water or aqueous materials may include water, aloe, or other materials. In some examples, the water based, alcohol-based material, or both, may constitute at lease about 50 wt % or more of the carrier composition. Additional examples of possible ointment, cream, or gel formulations are demonstrated below.

Similarly, representative topical balms, topical creams and topical gels containing the disclosed oils are discussed herein. However, other carrier bases (balm, cream, gel, liquid spray, carrier oils or other compositions) that are not described herein may also be used provided they include the disclosed manuka oil (or β-triketones from manuka oil) and palmarosa oil, CBD (oil or isolate) or patchouli oil-based compositions described herein.

In some embodiments, the carrier composition may be balm based and include one or more natural waxes that serve as a vehicle for the composition. Natural waxes provide a “green” alternative to synthetic- or petroleum-based derivatives. Natural waxes derived from plant-based materials or insect-based materials (e.g., bees' wax) provide vegan and/or vegetarian-friendly source materials. One or more natural waxes may act as emulsifiers that help keep the mixture of oils and other excipients from separating into aqueous and non-aqueous components. The waxes may also contribute to the sensory characteristics (e.g., touch and feel) of the topical composition. Suitable natural waxes may include, but are not limited to, beeswax, candelilla wax, camauba wax, jasmine wax, jojoba wax, laurel wax, mimosa wax, myrica fruit wax, orange wax, phyto wax, rapeseed wax, Rhus verniciflua peel wax (e.g., berry wax), Rhus succedanea fruit wax (e.g., Japan wax), rice bran wax, rose wax, soy wax, sunflower seed wax, tea wax, mixtures thereof and the like. Preferred waxes may include beeswax, Rhus succedanea fruit wax, and Rhus verniciflua peel wax. Beeswax, sometimes referred to as cera alba, is secreted by bees and can be sourced from a variety of places. Beeswax exhibits a relatively high melting point (e.g., about 61-65° C.) and remains workable at ambient to body temperatures. Beeswax has a silky texture and does not clog pores, both of which are desirable for skin applications. The beeswax may help to form a protective layer over skin to help prevent loss of moisture and help protect the skin from environmental conditions and external moisture. Beeswax also has the ability to act as an emulsifier in the topical composition.

Rhus succedanea fruit wax is obtained from the berries of the Rhus succedanea plant, which grows in Japan and China. Sometimes referred to as Japan wax, Rhus succedanea fruit wax has a relatively low melting point (e.g., about 45-55° C.) and acts as an emulsifier in the topical formulation to help prevent the separation of the oils and other excipients. Rhus succedanea fruit wax may also help to increase the plasticity of the topical composition to provide a semi-solid consistency that still allows for smooth and effective application of the composition as a topical balm.

Rhus verniciflua peel wax, also referred to as berry wax, is a soft wax derived from the peel of the fruit of the Rhus verniciflua plant. Berry wax has a relatively low melting point (e.g., about 48-55° C.). The wax is used to give a soft consistency that provides a cooling sensation with a silky feel and very desirable sensory characteristics when applied to the skin. Like Rhus succedanea fruit wax, Rhus verniciflua peel wax may also help to increase the plasticity of the topical composition to provide a semi-solid consistency that still allows for smooth and effective application of the composition as a topical balm.

The carrier composition may also include other optional excipients including, but not limited to, antioxidants, emollients, chelating agents (e.g., disodium EDTA), moisturizers, surfactants, emulsifiers, lubricants, natural oils, nutrients or vitamins, and the like. In many examples, a particular excipient may serve multiple functions within the topical composition. For example, tocopherol is both an antioxidant and vitamin that serves as a nutrient for the skin.

Optional excipients may include one or more antioxidants including, but not limited to, ascorbic acid or ascorbyl palmitate (vitamin C or a derivative thereof), tocopherol (vitamin E), or the like. Tocopherol is typically derived from vegetable oils and possesses exceptional skin protection properties. For example, tocopherol may be used to absorb UV rays and prevent UV induced free radical damage to the skin as well as prevent other free radical induced damage. Tocopherol has anti-inflammatory properties and helps moisturize the skin. Ascorbyl palmitate is a fat-soluble derivative of ascorbic acid that acts as a free radical scavenger. Unlike the water-soluble form (e.g., ascorbic acid), ascorbyl palmitate is able to enter the lipid cell membrane of the skin. Ascorbyl palmitate also supports immune cell activity and helps with the formation and maintenance of collagen within the skin. Tocopherol, ascorbyl palmitate, or ascorbic acid may be produced from natural and vegan-based sources. In preferred embodiments, the skin sanitizing composition includes tocopherol, ascorbyl palmitate, or both.

Other useful optional excipients include nutrients and vitamins. In some examples, the carrier composition may include one or more vitamins A, B3 (nicotinamide), C, D, E, or K, and minerals such as zinc. Preferred vitamins include vitamin A (e.g., retinol or retinol ester), vitamin C (e.g., ascorbyl palmitate or ascorbic acid), and vitamin E (tocopherol).

In some examples, the topical composition may also include at least vitamin A (e.g., retinol or retinol ester). The inclusion of retinol in the topical composition in conjunction with the other materials disclosed above may be useful for the treatment of acne. Inclusion of vitamin B3 and zinc in the topical composition in conjunction with the other materials disclosed above may also be useful for the treatment of acne.

Other useful optional excipients include natural oils, such as one or more plant oils, seed oils, essential oils, or the like. Natural oils may serve multiple functions within the topical composition such as acting as both a lubricant and emollient. Suitable natural oils may include, for example, Ricinus communis seed oil (castor oil), Simmondsia chinensis seed oil (jojoba oil) and Rosa canina (rosehip oil). Castor oil is a plant oil derived from the seeds of a Ricinus communis plant. Castor oil contains ricinoleic acid, which is a monounsaturated fatty acid that acts as a humectant. Castor oil helps moisturize the skin by preventing water loss, which in turn may help promote skin healing. Castor oil also has anti-inflammatory properties, antimicrobial activity, and can alleviate pain in some instances. Jojoba oil is a plant oil derived from the seeds of a Simmondsia chinensis plant. Jojoba oil acts as a moisturizer and emollient agent to improve the skin elasticity and suppleness, containing natural tocopherol to minimize oxidation. Jojoba oil behaves similarly to the natural oils of the skin and can penetrate the layers of the skin quickly to help promote healing without clogging pores or leaving a greasy feel to the skin. The combination of rosehip oil and manuka oil (or β-triketones extracted from manuka oil) assists in the production of collagen I and III that helps minimise fine lines and wrinkles, provides tensile strength and elasticity, improves skin hydration levels and plays a vital role in wound healing. Because castor, jojoba and rosehip oils are derived from plants, they provide vegan-based materials for the topical composition.

In some examples, the carrier composition may include one or more pre-formulated carrier compositions prepared by third party manufacturers. For example, the carrier composition may include one or more Kahl™ Jellies (e.g., VegoJelly 7036Plus), Kahl™ Bases (e.g., Natural Lip Care Base 7704), or Kahl™ Specialty Waxes (e.g., Veggiesoft Complex 6422), or similar products each available from Kahl GmbH & Co. KG of Germany. Such materials may include a blend of different excipients. Optionally, the carrier composition may also include preformulated, pharmaceutically acceptable Versapro™ cream, gel or lotion bases.

In some examples, the topical compositions may include one or more natural or synthetic pharmaceutically active excipients. Such pharmaceutically active excipients may be applied topically to treat a topical condition of the skin or be configured to be transmitted trans-dermally (e.g., transmitted into various layers of the skin or through the layers into the deep tissue or blood stream of the patient). In some examples, the pharmaceutically active excipient may be used to treat skin ailments such as, but not limited to, acne, bacterial infections, eczema, fungal infections, psoriasis, rashes, viral infections, or the like. In some such examples, the carrier composition may be formulated using pharmaceutically acceptable and compatible excipients. The described characteristics of manuka oil, palmarosa oil, and other ingredients may help to improve the efficacy of such pharmaceutically active excipients, limit the side effects of such pharmaceutically active excipients, or both.

As discussed above, the topical composition may consist substantially of, consist essentially of, or consist of natural or vegan based materials. However, several pharmaceutically active excipients may not be considered natural or vegan based excipients. In such embodiments, the topical composition serving as the vehicle for the pharmaceutically active excipients may nevertheless be characterized as consisting substantially or essentially of natural or vegan based materials.

The topical compositions disclosed may be used to treat one or more skin conditions including, but not limited to, inhibiting microbial growth on skin, treating skin lacerations or abrasions, treating acne, reducing the chance of infection after medical procedures such as those where the epidermis is breached by a needle, cannula, scalpel, or the like. The topical compositions may be applied directly to the afflicted area containing the skin condition. For example, the patient's skin may be initially cleaned around the site of the condition. The topical composition, such as a topical balm, cream or ointment may be applied liberally to the site and allowed to absorb into the skin. If additional protection is desired, a wound dressing or bandage may be applied over the topical composition, though the topical composition may also be used without such wound dressings. Additionally, or alternatively, the topical composition may be applied or incorporated into a wound dressing or bandage and then applied to the site containing the skin condition.

### EXAMPLES

The following examples are offered to aid in understanding of the above embodiments and are not to be construed as limiting the scope thereof.

### Example 1—Topical Balm

A mixture of the components shown in Table 1 is prepared to produce a topical balm.

TABLE 1

| | Amount |
|---|---|
| Component | (wt. %) |
| Manuka oil (or β-triketones from manuka oil) | 0.05-10 |
| Palmarosa oil, CBD oil, patchouli oil, or combinations | 0.1-10.0 |
| thereof | |
| Black seed oil, sandalwood oil, thyme oil, oregano oil, | 0-1.0 |
| cardamom oil, spearmint oil, German chamomile oil, | |
| α-bisabolol, β-caryophyllene, star anise oil, | |
| babchi or bakuchi oil, kanuka oil, Kakadu plum seed | |
| oil, gotukola extract or combinations thereof | |
| Ethylhexyl palmitate | 0-5.0 |
| Balm based carrier (e.g., Kahl VegoJelly 7036Plus) | balance |

### Example 2—Topical Antibacterial Ointment

A mixture of the components shown in Table 2 is prepared in the form of a topical ointment for the treatment of impetigo and other bacterial infections.

TABLE 2

| | Amount |
|---|---|
| Component | (wt. %) |
| Manuka oil (or β-triketones from manuka oil) | 0.1-10 |
| Palmarosa oil, CBD oil, palmarosa oil, patchouli oil, | 0.1-10.0 |
| or combinations thereof | |
| Black seed oil, sandalwood oil, thyme oil, oregano oil, | 0-1.0 |
| cardamom oil, spearmint oil, German chamomile oil, | |
| α-bisabolol, β-caryophyllene, star anise oil, | |
| babchi or bakuchi oil, kanuka oil, Kakadu plum seed | |
| oil, gotukola extract or combinations thereof | |
| Ointment base comprising 80% PEG 300 and 20% PEG 3350 | balance |

### Example 3—Cream for the Treatment of Dry Skin and Topical Inflammatory Skin Conditions

A mixture of the components shown in Table 3 is prepared in the form of a topical cream for the treatment of dry skin and topical inflammatory skin conditions such as eczema and psoriasis in adults and children.

TABLE 3

| | Amount |
|---|---|
| Component | (wt. %) |
| Manuka oil (or β-triketones from manuka oil) | 0.1-10.0 |
| CBD oil, palmarosa oil, patchouli oil, or combinations | 0.1-10.0 |
| thereof | |
| Black seed oil, sandalwood oil, thyme oil, oregano oil, | 0-1.0 |
| cardamom oil, spearmint oil, German chamomile oil, | |
| α-bisabolol, β-caryophyllene, star anise oil, | |
| babchi or bakuchi oil, kanuka oil, Kakadu plum seed | |
| oil, gotukola extract or combinations thereof | |
| Ointment base comprising PEG300 47.4%, PEG3350 12.6%, | balance |
| white soft paraffin 10%, stearyl alcohol 10%, propylene | |
| glycol 4.8%, sodium lauryl sulphate 0.4%, water 14.8% | |
| Total | 100 |

### Example 4—Topical Hydrophilic Ointment

A mixture of the components shown in Table 4 is prepared in the form of a topical ointment.

TABLE 4

| | Amount |
|---|---|
| Component | (wt. %) |
| Manuka oil (or β-triketones from manuka oil) | 0.05-10 |
| Palmarosa oil, CBD oil, patchouli oil, or combinations | 0.1-10.0 |
| thereof | |
| Black seed oil, sandalwood oil, thyme oil, oregano oil, | 0-1.0 |
| cardamom oil, spearmint oil, German chamomile oil, | |
| α-bisabolol, β-caryophyllene, star anise oil, | |
| babchi or bakuchi oil, kanuka oil, Kakadu plum seed | |
| oil, gotukola extract or combinations thereof | |
| Ointment base comprising water 37%, stearyl alcohol | balance |
| 25%, white soft paraffin 25%, propylene glycol 12%, | |
| sodium lauryl sulphate 1% | |

### Example 5—Topical Gel

A mixture of the components shown in Table 5 is prepared in the form of a topical gel.

TABLE 5

| | Amount |
|---|---|
| Component | (wt. %) |
| Manuka oil (or β-triketones from manuka oil) | 0.05-10 |
| CBD oil, palmarosa oil, patchouli oil, or combinations | 0.1-10.0 |
| thereof | |
| Black seed oil, sandalwood oil, thyme oil, oregano oil, | 0-1.0 |
| cardamom oil, spearmint oil, German chamomile oil, | |
| α-bisabolol, β-caryophyllene, star anise oil, | |
| babchi or bakuchi oil, kanuka oil, Kakadu plum seed | |
| oil, gotukola extract or combinations thereof | |
| Gel base 3 (aloe, allantoin, disodium EDTA, | balance |
| phenoxyethanol, triethylene glycol, water, optional | |
| binder (e.g., cellulose) | |

### Example 6—Topical Blemish Gel

A mixture of the components shown in Table 6 is prepared in the form of a topical gel for the treatment of skin blemishes including abrasions, acne, or other irritations.

TABLE 6

| | Amount |
|---|---|
| Component | (wt. %) |
| Manuka oil (or β-triketones from manuka oil) | 0.05-10.0 |
| CBD oil, palmarosa oil, patchouli oil, or combinations | 0.2-10.0 |
| thereof | |
| Black seed oil, sandalwood oil, thyme oil, oregano oil, | 0-1.0 |
| cardamom oil, spearmint oil, German chamomile oil, | |
| α-bisabolol, β-caryophyllene, star anise oil, | |
| babchi or bakuchi oil, kanuka oil, Kakadu plum seed | |
| oil, gotukola extract or combinations thereof | |
| Aqua (water) | 75-balance |
| Glycerin | 1-5 |
| Xantham Gum | 0.1-1 |
| Benzyl alcohol | 0.1-1 |
| Polysorbate 20 | 0.1-1 |
| Citrus limon peel oil | 0-0.1 |
| Retinyl palmitate | 0-0.1 |
| Dehydroacetic acid | 0-0.1 |
| Calendula officinalis flower extract | 0-0.1 |
| BHT (antioxidant) | 0-0.1 |
| Limonene | 0-0.1 |
| Citral | 0-0.1 |
| Linalool | 0-0.1 |
| Total | 100 |

The disclosed manuka oil β-triketone based blemish gel shows success as an acne spot treatment to prevent bacteria, makeup, and environmental debris from interfering with the skin's natural recovery process.

### Example 7-50% Minimum Inhibitory Concentrations (MIC₅₀) & Minimum Bactericidal Concentrations

The MIC₅₀ test evaluates the lowest concentration of a test substance that prevents 50% of the growth of bacteria or fungi. This is a measure of the antibacterial or anti-fungal activity of the test sample. MBC is the lowest concentration of an antibacterial agent required to kill a bacterium. Similarly, Minimum fungicidal concentration (MFC) is the lowest concentration of an antifungal agent required to kill a fungus or a yeast.

The disclosed topical compositions may be tested for MIC₅₀ and MBC using publicly available testing criteria. For example, test samples of the desired active compound can be prepared and serially diluted to give 11 or more different concentrations covering, for example, a 1000-fold range. For example, the test samples may include 50%-0.1% of a stock test sample (e.g., stock samples disclosed in Table 1) that are combined with select bacteria and fungi and incubated over a prescribed period of time. The MIC₅₀ is useful to determine the lowest concentration of each test sample (mixture of active materials) that inhibits 50% of the growth of the microbe as determined by measurement of OD_{650 nm} using a Versa Max 96 well plate reader. Measurement of cell concentration can be made at several time points.

Additional data Minimum Inhibitory Concentration (MIC) data on East Cape manuka oil and β-triketones provide further context. (Source: Plant & Food). MIC data is measured at the first sign of bacterial growth inhibition. These figures can be expected to be lower than MIC₅₀ data, though this is not always the case. Published MIC data on palmarosa oil is also included for further context, In Vitro Antimicrobial and Antioxidant Activities of Some Cymbopogon Species in Essential Oil-Bearing Grasses—The genus Cymbopogon, Khunkitti, W. (2010) Chapter 6, Edited by A. Akhila., Medicinal and Aromatic Plants—Industrial Profiles, CRC Press Taylor & Francis Group. Example stock sample solutions of active mixtures that show promising results and are suitable for MIC₅₀ and MBC testing are listed in Table 7.

TABLE 7
Stock solutions for MIC₅₀ and MBC testing

| Sample No. | Primary ingredients |
|---|---|
| 1 | 1.32% Manuka oil β-triketones, 0.33% black seed oil |
| 2 | 1.32% Manuka oil β-triketones, 0.68% palmarosa oil |
| 3 | 1.32% Manuka oil β-triketones, 0.68% sandalwood oil |
| 4 | 1.32% Manuka oil β-triketones, 0.68% thyme oil |
| 5 | 1.32% Manuka oil β-triketones, 0.68% patchouli oil |
| 6 | 1.32% Manuka oil β-triketones, 0.68% oregano oil |
| 7 | 1.32% Manuka oil β-triketones, 0.68% cardamom oil |
| 8 | 1.32% Manuka oil β-triketones, 0.68% spearmint oil |
| 9 | 1.32% Manuka oil β-triketones, 0.68% Q-bisabolol |
| 10 | 1.32% Manuka oil β-triketones, 0.68% gotukola extract |

The methodology for growing the different micro-organisms available for evaluation in the MIC₅₀ studies may be provided by the CLSI standards, Methods for Dilution Antimicrobial Susceptibility Tests for Bacteria that Grow Aerobically, Approved Standard, Ninth Edition, M07-A9, Vol. 32 No 2, January 2012; Methods for Antimicrobial Susceptibility Testing for Anaerobic Bacteria. Approved Standard, Ninth Edition, Mll-9, January 2018; and Performance Standards for Antifungal Susceptibility Testing of Filamentous Fungi. First Edition, M61, November 2017.

Microorganisms that may be tested include, but are not limited to, Acinetobacter baumannii; Candida albicans; Corynebacterium diphtheria; Cutibacterium acnes; Enterococcus faecium; Enterococcus faecalis; Escherichia coli; Proteus vulgaris; Pseudomonas aeruginosa; Staphylococcus aureus; methicillin-resistant Staphylococcus aureus (MRSA); Staphylococcus epidermidis; Streptococcus pyogenes; Trichophyton mentagrophytes; and Trichophyton rubrum, HSV 1 and Herpes zoster. The samples in Table 1, prepared in topical compositions as disclosed herein show promise against inhibiting growth of one or more of the above microorganisms.

Gram positive bacteria Staphylococcus aureus, methicillin-resistant Staphylococcus aureus (MRSA) and Streptococcus pyogenes cause impetigo, boils, abscesses and other common bacterial skin and wound infections.

Table 8 shows S. aureus MIC₅₀ and MBC data for selected samples, expressed in terms of the percentage of manuka oil or β-triketones from manuka oil.

TABLE 8
S. aureus MIC and MBC data

| Sample | MIC₅₀ (v/v) % | | MBC (v/v) % | |
|---|---|---|---|---|
| Manuka oil | 0.0156 | (av. of 2) | 0.0938 | (av. of 2) |
| Manuka oil (MIC)** | 0.078-0.125 | | | |
| Manuka oil β-triketones | 0.0051 | (av. of 2) | 0.0825 | (av. of 2) |
| Manuka oil β-triketones (MIC)** | 0.01-0.08 | | | |
| Manuka oil β-triketones + blackseed oil | 0.0026 | (av. of 3) | 0.055 | (av. of 3) |
| Manuka oil β-triketones + palmarosa oil | 0.0015 | (av. of 3) | 0.0275 | (av. of 3) |

| | | |
|---|---|---|
| Manuka oil β-triketones + sandalwood oil | 0.0103 | 0.0206 |
| Manuka oil β-triketones + thyme oil | 0.0103 | 0.0206 |
| Manuka oil β-triketones + patchouli oil | 0.0052 | >0.66 |
| Palmarosa oil (MIC)* | 0.1% | — |
| Tea tree oil (2%) | 2 | 2 |
| Manuka honey UMF 20+ (10%) | 0.626 | >10% |

*Source: Khunkitti (2010)
**Source: Plant & Food

Table 9 shows MRSA MIC₅₀ and MBC data for selected samples.

TABLE 9
MRSA MIC₅₀ and MBC data

| | | MBC |
|---|---|---|
| Sample | MIC₅₀ (v/v) % | (v/v) % |
| Manuka oil (MIC)** | 0.020-0.060 | — |
| Manuka oil β-triketones (MIC)** | 0.0035-0.010 | — |
| Manuka oil β-triketones + blackseed oil | 0.0052 | 0.0413 |
| Manuka oil β-triketones + palmarosa oil | 0.0026 | 0.0103 |
| Manuka oil β-triketones + sandalwood oil | 0.0206 | 0.0413 |
| Manuka oil β-triketones + oregano oil | 0.0206 | 0.0413 |
| Manuka oil β-triketones + cardamom oil | 0.0052 | 0.0103 |
| Tea tree oil (2%) | >2 | >2 |

**Source: Plant & Food

Table 10 shows S. pyogenes MIC₅₀ and MBC data for selected samples.

TABLE 10
S. pyogenes MIC₅₀ and MBC data

| Sample | MIC₅₀ (v/v) % | MBC (v/v) % |
|---|---|---|
| Manuka oil (MIC)** | 0.125 | — |
| Manuka oil β-triketones (MIC)** | 0.0625 | — |
| Manuka oil β-triketones + blackseed oil | 0.0103 | 0.165 |
| Manuka oil β-triketones + palmarosa oil | 0.0051 | 0.0206 |
| Manuka oil β-triketones + thyme oil | 0.0051 | >0.66 |
| Manuka oil β-triketones + oregano oil | 0.0051 | 0.0413 |
| Manuka oil β-triketones + spearmint oil | 0.0103 | 0.0413 |
| Tea tree oil (2%) | 0.25% | 0.5% |

**Source: Plant & Food

Gram positive bacteria Cutibacterium acnes and Staphylococcus epidermidis bacteria are associated with inflammatory acne. Staphylococcus epidermidis is also found in surgical site infections.

Table 11 shows C. acnes MIC₅₀ and MBC data for selected samples

TABLE 11
C. acnes MIC₅₀ and MBC data

| | | MBC |
|---|---|---|
| Sample | MIC₅₀ (v/v) % | (v/v) % |
| Manuka oil (MIC)** | 0.07 | — |
| Manuka oil β-triketones + blackseed oil | 0.0103 | >0.66 |
| Manuka oil β-triketones + palmarosa oil | <0.0006 | 0.0052 |
| Manuka oil β-triketones + sandalwood oil | 0.0206 | 0.0825 |
| Manuka oil β-triketones + thyme oil | 0.0052 | 0.0206 |
| Manuka oil β-triketones + patchouli oil | 0.0206 | 0.0413 |
| Tea tree oil (2%) | 2 | >2 |

**Source: Plant & Food

Table 12 shows S. epidermidis MIC₅₀ and MBC data for selected samples.

TABLE 12
S. Epidermidis MIC₅₀ and MBC data

| | | MBC |
|---|---|---|
| Sample | MIC₅₀ (v/v) % | (v/v) % |
| Manuka oil (MIC)** | 0.050-0.078 | — |
| Manuka oil β-triketones (MIC)** | 0.010 | — |
| Manuka oil β-triketones + blackseed oil | 0.0103 | 0.0413 |
| Manuka oil β-triketones + palmarosa oil | 0.0013 | 0.0206 |
| Manuka oil β-triketones + sandalwood oil | 0.0206 | 0.0413 |
| Manuka oil β-triketones + oregano oil | 0.0206 | 0.0413 |
| Manuka oil β-triketones + cardamom oil | 0.0052 | 0.0206 |
| Tea tree oil (2%) | >2 | >2 |

**Source: Plant & Food

Candida albicans cause oral and vaginal thrush, jock itch, diaper rash, nail fungus and other infections. Table 13 shows C. albicans MIC₅₀ and MFC data for selected samples.

TABLE 13
C. albicans MIC₅₀ and MFC data

| | | MFC |
|---|---|---|
| Sample | MIC₅₀ (v/v) % | (v/v) % |
| Manuka oil (MIC)** | 0.625->2.00 | — |
| Manuka oil β-triketones (MIC)** | 0.040-<1.00 | — |
| Manuka oil β-triketones + blackseed oil | 0.66 | >0.66 |
| Manuka oil β-triketones + palmarosa oil | 0.0825 | 0.33 |
| Manuka oil β-triketones + sandalwood oil | 0.66 | >0.66 |
| Manuka oil β-triketones + thyme oil | 0.165 | >0.66 |
| Manuka oil β-triketones + cardamom oil | 0.66 | 0.66 |
| Tea tree oil (2%) | 2 | 2 |
| Palmarosa oil (MIC)* | 0.1 | — |

*Source: Khunkitti (2010)
**Source: Plant & Food

Trichophyton mentagrophytes and Trichophyton rubrum cause athlete's foot, jock itch, ring worm and other fungal infections.

Table 14 shows T. mentagrophytes MIC₅₀ and MFC data for selected samples.

TABLE 14
T. mentagrophytes MIC₅₀ and MFC data

| Sample | MIC₅₀ (v/v) % | MFC (v/v) % |
|---|---|---|
| Manuka oil | 0.06 | — |
| Manuka oil (MIC)** | 0.050-0.080 | — |
| Manuka oil β-triketones | >0.33 | — |
| Manuka oil β-triketones + blackseed oil | 0.66 | >0.66 |
| Manuka oil β-triketones + palmarosa oil | 0.06 (av. of 2) | >0.66 |
| Manuka oil β-triketones + thyme oil | 0.0413 | 0.66 |
| Manuka oil β-triketones + oregano oil | 0.0413 | >0.66 |
| Tea tree oil (2%) | 1 | 2 |

**Source: Plant & Food

Table 15 shows T. rubrum MIC₅₀ and MFC data for selected samples.

TABLE 15
T. rubrum MIC₅₀ and MFC data

| Sample | MIC₅₀ (v/v) % | MFC (v/v) % |
|---|---|---|
| Manuka oil (MIC)** | <0.039-0.30 | — |
| Manuka oil β-triketones (MIC)** | 0.32 | — |
| Manuka oil β-triketones + blackseed oil | 0.66 | >0.66 |
| Manuka oil β-triketones + palmarosa oil | 0.0825 | >0.66 |
| Manuka oil β-triketones + thyme oil | 0.0103 | 0.165 |
| Manuka oil β-triketones + oregano oil | 0.0413 | 0.0825 |
| Tea tree oil (2%) | 1 | >2 |

**Source: Plant & Food

Gram positive bacteria Corynebacterium diphtheriae, Enterococcus faecalis, Enterococcus faecium and cause a wide range of potentially serious skin and wound infections. Corynebacterium minutissimum causes the unpleasant condition of erythrasma.

Table 16 shows C. diphtheriae MIC₅₀ and MBC data for selected samples.

TABLE 16
C. diphtheriae MIC₅₀ and MBC data

| Sample | MIC₅₀ (v/v) % | MBC (v/v) % |
|---|---|---|
| Manuka oil (MIC)** | 0.050 | — |
| Manuka oil β-triketones (MIC)** | 0.020 | — |
| Manuka oil β-triketones + blackseed oil | 0.0206 | >0.66 |
| Manuka oil β-triketones + palmarosa oil | 0.0103 | >0.66 |
| Manuka oil β-triketones + thyme oil | 0.0103 | >0.66 |
| Manuka oil β-triketones + oregano oil | 0.0103 | >0.66 |
| Manuka oil β-triketones + Q-bisabolol | 0.0052 | >0.33 |
| Tea tree oil (2%) | 0.25 | >2 |

**Source: Plant & Food

Table 17 shows E. faecalis MIC₅₀ and MBC data for selected samples.

TABLE 17
E. faecalis MIC₅₀ and MBC data

| | | MBC |
|---|---|---|
| Sample | MIC₅₀ (v/v) % | (v/v) % |
| Manuka oil β-triketones + blackseed oil | 0.0413 | 0.0825 |
| Manuka oil β-triketones + palmarosa oil | 0.0206 | 0.0413 |
| Manuka oil β-triketones + patchouli oil | 0.0413 | 0.0413 |
| Manuka oil β-triketones + oregano oil | 0.0206 | 0.0413 |
| Manuka oil β-triketones + cardamom oil | 0.0206 | 0.0825 |
| Palmarosa oil (MIC)* | 0.25 | — |
| Tea tree oil (2%) | 0.25 | >2 |

*Source: Khunkitti (2010)

Table 18 shows E. faecium MIC₅₀ and MBC data for selected samples.

TABLE 18
E. faecium MIC₅₀ and MBC data

| | | MBC |
|---|---|---|
| Sample | MIC₅₀ (v/v) % | (v/v) % |
| Manuka oil (MIC)** | 0.050 | — |
| Manuka oil β-triketones (MIC)** | 0.010 | — |
| Manuka oil β-triketones + blackseed oil | 0.0206 | 0.66 |
| Manuka oil β-triketones + palmarosa oil | 0.0206 | 0.0413 |
| Manuka oil β-triketones + patchouli oil | 0.0103 | 0.0206 |
| Manuka oil β-triketones + oregano oil | 0.0206 | 0.0413 |
| Manuka oil β-triketones + cardamom oil | 0.0206 | 0.0413 |
| Tea tree oil (2%) | 1% | >2 |

**Source: Plant & Food

Table 19 shows C. minutissimum MIC₅₀ and MBC data for selected samples.

TABLE 19
C. minutissimum MIC₅₀ and MBC data

| Sample | MIC₅₀ (v/v) % | MBC (v/v) % |
|---|---|---|
| Manuka oil (MIC)** | 0.05 | — |
| Manuka oil β-triketones (MIC)** | 0.02 | — |
| Manuka oil β-triketones + blackseed oil | 0.0206 | 0.165 |
| Manuka oil β-triketones + palmarosa oil | 0.0206 | 0.0206 |
| Manuka oil β-triketones + thyme oil | 0.0206 | 0.0825 |
| Manuka oil β-triketones + oregano oil | 0.0103 | 0.0825 |
| Manuka oil β-triketones + Q-bisabolol | 0.0052 | 0.0413 |
| Tea tree oil (2%) | 0.125% | 1% |

**Source: Plant & Food

Gram negative bacteria Escherichia coli, Proteus vulgaris and Acinetobacter baumannii can cause serious skin, wound and surgical site infections.

Table 20 shows E. coli MIC₅₀ and MBC data for selected samples.

TABLE 20
E. coli MIC₅₀ and MBC data

| | | MBC |
|---|---|---|
| Sample | MIC₅₀ (v/v) % | (v/v) % |
| Manuka oil (MIC)** | >2.00 | — |
| Manuka oil β-triketones (MIC)** | 0.320->2.00 | — |
| Manuka oil β-triketones + blackseed oil | 0.33 | >0.66 |
| Manuka oil β-triketones + palmarosa oil | 0.165 | 0.165 |
| Manuka oil β-triketones + thyme oil | 0.0825 | 0.0825 |
| Manuka oil β-triketones + oregano oil | 0.0413 | 0.0413 |
| Manuka oil β-triketones + gotukola extract | >0.66 | >0.66 |
| Tea tree oil (2%) | 0.25 | 0.5 |
| *Palmarosa oil (MIC)* | 0.2 | — |

*Khunkitti (2010)
**Source: Plant & Food

Table 21 shows P. vulgaris MIC₅₀ and MBC data for selected samples.

TABLE 21
P. vulgaris MIC₅₀ and MBC data

| | | MBC |
|---|---|---|
| Sample | MIC₅₀ (v/v) % | (v/v) % |
| Manuka oil (MIC)** | 0.500->2.00 | — |
| Manuka oil β-triketones (MIC)** | 0.32 | — |
| Manuka oil β-triketones + blackseed oil | 0.165 | >0.66 |
| Manuka oil β-triketones + palmarosa oil | 0.0825 | 0.33 |
| Manuka oil β-triketones + sandalwood oil | 0.165 | >0.66 |
| Manuka oil β-triketones + thyme oil | 0.0413 | 0.0825 |
| Manuka oil β-triketones + cardamom oil | 0.165 | 0.66 |
| Tea tree oil (2%) | 0.25 | 0.5 |

**Source: Plant & Food

Table 22 shows P. aeruginosa MIC₅₀ and MBC data for selected samples.

TABLE 22
P. aeruginosa MIC₅₀ and MBC data

| | | MBC |
|---|---|---|
| Sample | MIC₅₀ (v/v) % | (v/v) % |
| Manuka oil (MIC)** | 0.850−>2.00 | — |
| Manuka oil β-triketones (MIC)** | 0.320 −>2.00 | — |
| Manuka oil β-triketones + blackseed oil | 0.66 | >0.66 |
| Manuka oil β-triketones + palmarosa oil | 0.66 | >0.66 |
| Manuka oil β-triketones + sandalwood oil | >0.66 | >0.66 |
| Manuka oil β-triketones + cardamom oil | 0.66 | >0.66 |
| Manuka oil β-triketones + gotukola extract | >0.66 | >0.66 |
| Tea tree oil (2%) | >2 | >2 |
| Palmarosa oil (MIC)* | >2 | — |

*Khunkitti (2010)
**Source: Plant & Food

Table 23 shows A. baumannii MIC₅₀ and MBC data for selected samples.

TABLE 23
A. baumannii MIC₅₀ and MBC data

| | | MBC |
|---|---|---|
| Sample | MIC₅₀ (v/v) % | (v/v) % |
| Manuka oil β-triketones + blackseed oil | >0.66 | >0.66 |
| Manuka oil β-triketones + palmarosa oil | 0.33 | >0.66 |
| Manuka oil β-triketones + cardamom oil | >0.66 | >0.66 |
| Manuka oil β-triketones + oregano oil | >0.66 | >0.66 |
| Manuka oil β-triketones + thyme oil | 0.33 | >0.66 |
| Tea tree oil | >2 | >2 |
| Palmarosa oil (MIC)* | 0.1 | — |

*Khunkitti (2010)

Known antibiotic and antifungal samples can also be tested as a comparative sample and control. Such antimicrobials can include, but are not limited to Amoxicillin, Cefalotin, Colistin, Erythromycin, Itraconazole, Metronidazole, and Penicillin.

Manuka β-triketones and CBD isolate demonstrate synergistic antimicrobial properties with Cutibacterium acnes. Table 24 shows MIC₅₀ data for selected samples.

TABLE 24
C. acnes studies with manuka Oil β-triketones and CBD

| Sample | MIC₅₀ (v/v) % |
|---|---|
| 1% Manuka oil β-triketones | 0.25% Manuka oil β-triketones |
| 0.05% CBD isolate | 0.006% CBD |
| 1% Manuka oil β-triketones + | 0.006% Manuka oil β-triketones + |
| 0.05% CBD | 0.003% CBD |

### Example 8—Therapeutic Concentrations of Antimicrobial Compositions

The data in EXAMPLE 7, is a useful guide for formulators and clinicians in determining the choice and approximate dosage of antimicrobials in the treatment of patients.

Table 25 summarises indicative antimicrobial therapeutic dosages based on the MBC data in EXAMPLE 7. The suggested upper limits are supported by Human Repeat Insult Patch Testing (HRIPT) of the products at a single concentration point (2%) with 50 or more people.

TABLE 25
Indicative therapeutic antimicrobial dosages

| | Gram +ve | Gram −ve | Fungi & |
|---|---|---|---|
| Product | Bacteria | Bacteria | Yeast |
| Manuka oil | 0.1 to 2% | 1-2% | 0.1 to 2% |
| β-Triketones | 0.05 to 2% | 1-2% | 1 to 2% |
| 3k+ ™ (β-triketones + | 0.05 to 2% | 1-2% | 1 to 2% |
| blackseed oil) | | | |
| ECMT-154 ™ (β-triketones + | 0.02 to 2% | 0.5 to 2% | 0.1 to 2% |
| palmarosa oil) | | | |

### Example 9—Zone of Inhibition Testing of Topical Formulations Containing Manuka Oil β-Triketones and Palmarosa Oil

Six different topical carrier base formulations were developed and tested in order to identify the antimicrobial effectiveness of manuka oil β-triketones and palmarosa oil incorporated into these formulations.

The technique of Zone of Inhibition was used to test these topical formulations. Test samples were incubated with the bacteria and the zone of inhibition for each test sample was measured after 24 hours of incubation. In the Zone of Inhibition test (also known as the Disk Diffusion Antibiotic Sensitivity test or the Kirby-Bauer test), a thin film of bacteria was applied to a nutrient enriched agar plate and then subjected to the various antibiotic ointments. The zone of inhibition refers to a circular area around the antibiotic test sample in which the bacteria colonies do not grow. The zone of inhibition can be used to measure the susceptibility of bacteria towards a given antimicrobial product, such that the size of the zone of inhibition may be used as a representation of the antimicrobial effectiveness of a particular composition. More specifically, the diameter of the zone of inhibition is indicative of the efficacy of the specific antimicrobial composition.

The first step in this process is to ascertain the appropriate concentration of therapeutic ingredients based on the relative amounts of β-triketones and palmarosa oil. Table 26 lists the four antimicrobial compositions (therapeutic extracts) that were tested and their Zone of Zone of Inhibition results against S. aureus and S. pyogenes.

TABLE 26
Zone of Inhibition results against S. aureus and S. pyogenes.

| | | Mean |
|---|---|---|
| | | Diameter |
| Pathogen | Therapeutic Extract Composition | (mm) |
| Staphylococcus | 0.66% β-triketones + 0.34% palmarosa oil | 12.8 ± 0.5 |
| aureus | 0.66% β-triketones + 1% palmarosa oil | 12.8 ± 0.3 |
| | 1% β-triketones + 1% palmarosa oil | 13.3 ± 0.4 |
| | 2% β-triketones + 2% palmarosa oil | 16.7 ± 0.2 |
| | Penicillin reference (0.5 μg/ml) | 16.4 ± 0.2 |
| Streptococcus | 0.66% β-triketones + 0.34% palmarosa oil | 19.8 ± 0.3 |
| pyogenes | 0.66% β-triketones + 1% palmarosa oil | 21.0 ± 0.6 |
| | 1% β-triketones + 1% palmarosa oil | 22.2 ± 0.4 |
| | 2% β-triketones + 2% palmarosa oil | 22.5 ± 0.8 |
| | Penicillin reference (0.5 μg/ml) | 21.4 ± 0.6 |

These results show that the topical composition containing 2% β-triketones and 2% palmarosa oil demonstrate greater effectiveness than the penicillin standard against S. aureus. These results also show that the formulation with 1% β-triketones and 1% palmarosa oil and 2% β-triketones and 2% palmarosa oil proved greater effectiveness than penicillin against S. pyogenes.

The formulation with 2% β-triketones and 2% palmarosa proved the strongest effectiveness against both S. aureus and S. pyogenes and was therefore used for further testing in the topical base formulation comparison tests of Example 11.

### Example 10—Carrier Base Composition Development

Table 27 lists the six topical carrier base formulations that were tested to explore the effectiveness of topical compositions comprising manuka oil (more specifically, manuka oil β-triketones) and palmarosa oil compounded with these carrier bases.

TABLE 27
Six topical base formulations for antimicrobial compositions

| # | Carrier Base | Ingredients |
|---|---|---|
| 1 | CosCo Hydrophilic | Water, betaine, caprylic/capric triglyceride, cetearyl alcohol, |
| | Ointment | propanediol, sodium lactate, coco-caprylate, glycerine, |
| | | sodium stearoyl glutamate, lactic acid, dimethicone, sodium |
| | | phytate, phenoxyethanol, sodium lauryl sulphate, xanthan |
| | | gum, ethylhexylglycerin |
| 2 | CosCo Kahl Vegojelly | Ricinus communis (castor) seed oil, hydrogenated Rhus |
| | Balm | verniciflua (varnish tree) peel wax, rhus succedanea fruit |
| | | wax, ascorbyl palmitate, tocopherol, ethyl hexyl palmitate, |
| | | Simmondsia chinensis (jojoba) seed oil, cera alba |
| 3 | Optimus Cream 1 | Water, stearyl alcohol, white soft paraffin, propylene glycol, |
| | | sodium lauryl sulphate, benzyl alcohol, lactic acid |
| 4 | Optimus Cream 3 | Water, stearyl alcohol, propylene glycol, glycerol, sodium |
| | | lauryl sulphate, white soft paraffin, ethylcellulose |
| 5 | Optimus Versapro gel | Water, allantoin, aloe, disodium EDTA, phenoxyethanol, |
| | | PVM/MA deacdiene cross-polymer, triethylene glycol |
| 6 | Optimus PEG base | PEG 300, PEG 3350 |

### Example 11—Base Composition Efficacy Test

The base carrier formulations of Example 10 were tested in conjunction with manuka oil and palmarosa oil (e.g., at least 2% β-triketones+2% palmarosa oil based on Example 9) using the technique of Zone of Inhibition. Test samples were incubated with the bacteria and the zone of inhibition for each test sample was measured after 24 hours of incubation. Various formulations were developed and tested against three reference standards (Fucidin® antibiotic cream with 2% fusidic acid, Betadine® with 10% povidone iodine and Crystaderm® with 1% hydrogen peroxide) to identify the antimicrobial effectiveness of each of the formulations against S. aureus.

Table 28 lists the topical compositions that were tested in order to identify the optimal carrier base, and the resulting zone of inhibition data.

TABLE 28
Zone of inhibition data on topical compositions

| | | Zone of | |
|---|---|---|---|
| Base | Active ingredients | Inhibition (mm) | Effectiveness |
| 1 | 2% β-triketones + 2% palmarosa oil | 11.0 ± 0.7 | Minor |
| 2 | 4% β-triketones + 4% palmarosa oil | 14.7 ± 0.1 | Minor |
| 3 | 2% β-triketones + 2% palmarosa oil | 21.1 ± 0.5 | Medium |
| 4 | 2% β-triketones + 2% palmarosa oil | 18.7 ± 0.4 | Medium |
| 5 | 2% β-triketones + 2% palmarosa oil | 23.2 ± 0.7 | Medium |
| 6 | 2% β-triketones + 2% palmarosa oil | 40 ± 2 | High |
| Fucidin ® | 2% fusidic acid antibiotic cream | 39.9 ± 0.2 | High |
| | | 38 ± 1 | |
| | | 39.7 ± 0.5 | |
| Crystaderm ® | 1% hydrogen peroxide cream | 36.8 ± 0.5 | High |
| Betadine ® | 10% povidone iodine ointment | 12.4 ± 0.2 | Minor |

Based on the results in Table 28, the Optimus PEG base carrier (Base 6, Table 21) appeared to be most effective base carrier formulation against S. aureus and likely to be most suited for therapeutically effective impetigo treatment.

Further testing was conducted using the Optimus PEG base carrier formulation containing 2% manuka β-triketones and 2% palmarosa oil to examine the effectiveness of this formulation and reference standards against S. pyogenes and MRSA. These results are summarised in Table 29.

TABLE 29
Antimicrobial Effectiveness of 2% β-triketones + 2% palmarosa
oil in PEG Base against S. pyogenes and MRSA

| Base | Active ingredients | Zone of Inhibition (mm) | Pathogen |
|---|---|---|---|
| 6 (PEG base) | 2% β-triketones + | 33 ± 1 | S. pyogenes |
| | 2% palmarosa oil | | |
| Fucidin ® | 2% fusidic acid | 20.8 ± 0.2 | S. pyogenes |
| Crystaderm ® | 1% hydrogen peroxide | 16.0 ± 0.2 | S. pyogenes |
| Betadine ® | 10% povidone iodine | 20.0 0.3 | S. pyogenes |
| 6 (PEG base) | 2% β-triketones + | >45 (exceeded prmtr) | MRSA |
| | 2% palmarosa oil | | |
| Fucidin ® | 2% fusidic acid | >45 (exceeded prmtr) | MRSA |
| Crystaderm ® | 1% hydrogen peroxide | 35.9 ± 0.1 | MRSA |
| Betadine ® | 10% povidone iodine | 12.8 ± 0.5 | MRSA |

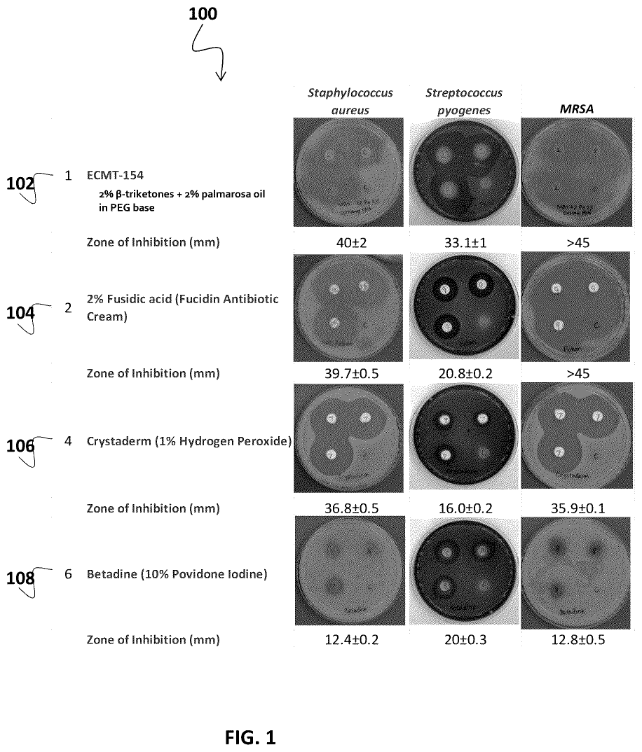

S. aureus, MRSA and S. pyogenes zone of inhibition images for the ointment comprising 2% β-triketones and 2% palmarosa oil in the PEG base carrier (also known as ECMT-154 PEG Ointment), Betadine® and Crystaderm® are shown in FIG. 1. FIG. 1 is a table 100 demonstrating antimicrobial data for ECMT-154 PEG ointment and references, according to embodiments of the present disclosure.

The results in FIG. 1 confirm that the topical composition containing 2% (3-triketones+2% palmarosa oil and PEG base carrier ointment (ECMT-154 PEG) is highly effective against S. aureus and MRSA, shown in row 102, particularly compared to the reference standards. ECMT-154 PEG was more effective than Crystaderm® (1% hydrogen peroxide) cream, shown in row 106, and significantly more effective than Betadine® (10% povidone iodine) ointment, shown in row 108, for both pathogens while also significantly more effective than Fucidin® (2% fusidic acid) cream, shown in row 104, against S. pyogenes. The tests for both the ECMT-154 PEG and Fucidin® cream demonstrated that both products were highly effective against MRSA but could not be directly compared as both ointments produced zone diameters that exceeded possible measurement.

Table 30 summarises comparative data for 2% β-triketones+2% palmarosa oil in a PEG carrier base (ECMT-154 PEG ointment) against reference standards Fucidin®, Betadine® and Crystaderm®.

TABLE 30
Comparative data on ECMT-154 PEG ointment and reference standards

| | ECMT-154 PEG vs | ECMT-154 PEG vs | ECMT-154 PEG vs |
|---|---|---|---|
| Pathogen | Fucidin ® | Betadine ® | Crystaderm ® |
| S. aureus | — | ECMT-154 PEG is | ECMT-154 PEG is |
| | | 223% more effective | 9% more effective |
| S. pyogenes | ECMT-154 PEG is | ECMT-154 PEG is | ECMT-154 PEG is |
| | 59% more effective | 66% more effective | 107% more effective |
| MRSA | — | ECMT-154 PEG is | ECMT-154 PEG is |
| | | 252% more effective | 25% more effective |

### Example 12—Treatment of Impetigo and Other Bacterial Infections

Topical compositions are prepared including Leptospermum scoparium oil (manuka oil) or β-triketones extracted from manuka oil, Cymbopogon martinii (palmarosa oil), and a carrier composition that optionally includes polyethylene glycol. The topical composition is prepared and tested against skin ailments such as impetigo and wound infections. The formulation demonstrates a therapeutic effectiveness in the treatment of these infections.

### Example 13—Zone of Inhibition Testing of Acne Pathogens

Zone of inhibition testing was undertaken to compare the effectiveness of the topical composition containing 2% β-triketones+2% palmarosa oil in three different bases (3, 5 and 6 in Example 10) with standard acne treatments Benzac (2.5% benzoyl peroxide) and Clearasil® (1.9% salicylic acid) against Cutibacterium acnes and Staphylococcus epidermidis.

The zone of inhibition test results for topical composition containing 2% β-triketones+2% palmarosa oil and Optimus Cream 1 (ECMT-154 CREAM), 2% β-triketones+2% palmarosa oil and Optimus Versapro Gel (ECMT-154 GEL) and 2% β-triketones+2% palmarosa oil and PEG base carrier ointment (ECMT-154 PEG, sometimes known as Tripalma), Benzac and Clearasil® are summarised in Table 31.

TABLE 31
Zone of inhibition results for topical compositions
- C. acnes & S. epidermidis

| | | Zone of | |
|---|---|---|---|
| | | Inhibition | |
| Product | Active ingredients | (mm) | Pathogen |
| ECMT-154 | 2% β-triketones + | 21.5 ± 0.3 | C. acnes |
| Cream (base 3) | 2% palmarosa oil | | |
| ECMT-154 | 2% β-triketones + | 27 ± 1 | C. acnes |
| Gel (base 5) | 2% palmarosa oil | | |
| ECMT-154 | 2% β-triketones + | 23.9 ± 0.5 | C. acnes |
| PEG (base 6) | 2% palmarosa oil | | |
| Benzac | 2.5% benzoyl peroxide | 13.6 ± 0.2 | C. acnes |
| Clearasil ® | 1.9% salicylic acid | 9.9 ± 0.1 | C. acnes |
| ECMT-154 | 2% β-triketones + | 27.8 ± 0.9 | S. epidermidis |
| Cream (base 3) | 2% palmarosa oil | | |
| ECMT-154 | 2% β-triketones + 2% | 34 ± 2 | S. epidermidis |
| Gel (base 5) | palmarosa oil | | |
| ECMT-154 | 2% β-triketones + 2% | >45 | S. epidermidis |
| PEG (base 6) | palmarosa oil | | |
| Benzac | 2.5% benzoyl peroxide | 29.6 ± 0.8 | S. epidermidis |
| Clearasil ® | 1.9% salicylic acid | 11.6 ± 0.7 | S. epidermidis |

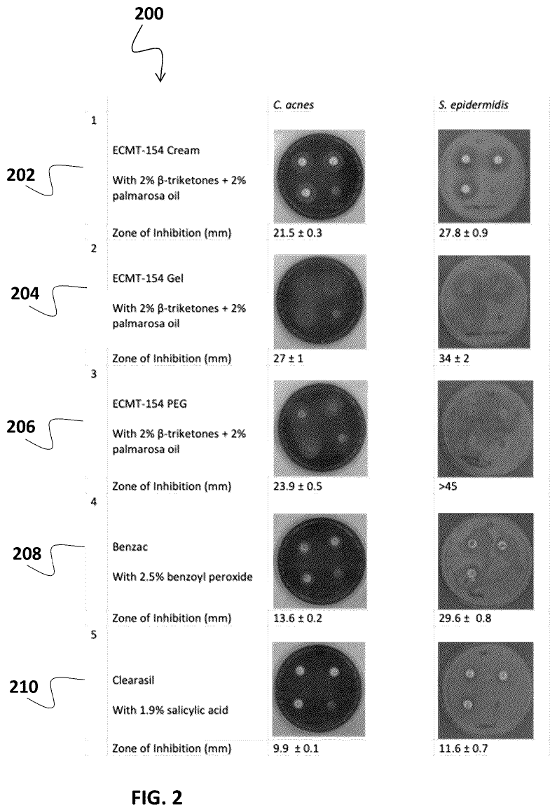

C. acnes and S. epidermidis zone of inhibition images for the following topical compositions are summarised in FIG. 2.

FIG. 2 is a table 200 of demonstrating the effect of ECMT-154 topical compositions and references against C. acnes and S. epidermidis. Table 200 shows results for 2% β-triketones+2% palmarosa oil and Optimus Cream 1 (ECMT-154 cream) at 202, 2% (3-triketones+2% palmarosa oil and Optimus Versapro gel (ECMT-154 gel) at 204, 2% (3-triketones+2% palmarosa oil in PEG base (ECMT-154 PEG or Tripalma) at 206, Benzac at 208, and Clearasil® at 210. The results in Table 31 and FIG. 2 confirm that the topical composition containing 2% β-triketones+2% palmarosa oil in a PEG base carrier (ECMT-154 PEG or Tripalma ointment), Versapro gel based carrier (ECMT-154 Gel) and a cream based carrier (ECMT-154 cream) are highly effective against C. acnes, particularly compared to the reference standards Benzac and Clearasil®. Of all the products tested, ECMT-154 Gel was most effective against C. acnes.

ECMT-154 PEG and ECMT-154 Gel were also more effective than the reference standards against S. epidermidis. Of the products tested, ECMT-154 PEG ointment was most effective against S. epidermidis.

### Example 14—Wound Healing Study

Aspects of wound healing include debridement, both pro- and anti-inflammation, cell proliferation and migration, angiogenesis, and extracellular matrix synthesis. Any product or preparation that can promote one or more of these stages may be able to accelerate wound healing. It is possible to investigate the potential of test samples to stimulate human skin cell proliferation and migration as well as their production of matrix constituents that are fundamental to the integrity and structure of skin. The effect on the healing rate was determined from digital photographs of the healing wounds taken at regular intervals. Test sample concentrations were selected based on initial fibroblast toxicity studies.

Table 32 shows the effect of test samples on rate of wound closure. The combination of β-triketones and palmarosa oil demonstrated the greatest wound healing effect.

TABLE 32
Effect of Test Samples on Rate of Wound Closure With Skin Fibroblasts

| | | 24 hours | 48 hours | 72 hours |
|---|---|---|---|---|
| # | Test Sample | % of Original Wound Size | | |
| | Control | 54.49 | 33.19 | 11.58 |
| 1 | 0.05% β-triketones | 95.75 | 98.22 | — |
| 2 | 0.003% β-triketones + 0.0015% palmarosa oil | 57.82 | 29.39 | 7.97 |
| 3 | 0.013% β-Triketones + 0.006% Thyme oil | 86.92 | 85.93 | — |
| 4 | 0.003% β-Triketones + 0.0015% oregano oil | 70.90 | 40.22 | 21.88 |
| 5 | 0.006% β-triketones + 0.003% sandalwood oil | 79.09 | 67.60 | — |
| 6 | 0.05% β-triketones + 0.1% rosehip oil | 99.77 | 100.02 | — |
| 7 | 0.05% β-triketones + 1% rosehip oil | 92.69 | 90.93 | 96.95 |

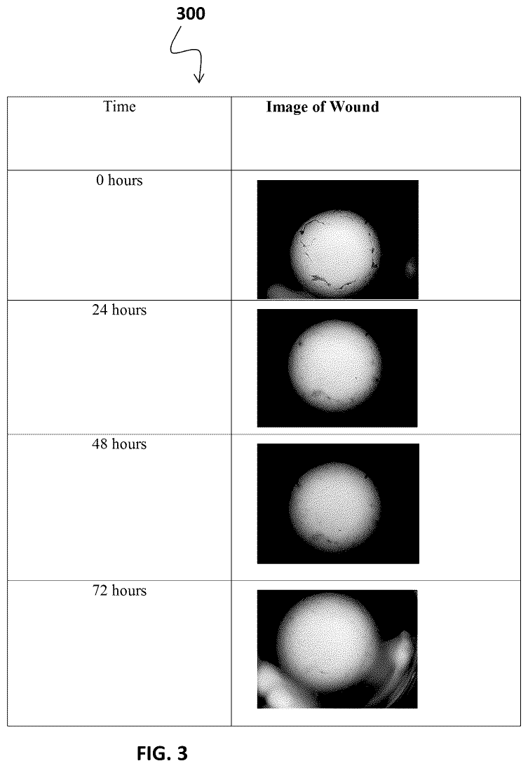

FIG. 3 summarises the photographs demonstrating the wound healing effect of a therapeutic extract comprising manuka oil β-triketones and palmarosa oil. FIG. 3 is a table 300 demonstrating the wound healing effect of manuka oil β-triketones and palmarosa oil, according to embodiments of the present disclosure.

### Example 15—Skin Barrier Repair Properties of Therapeutic Extracts Containing Manuka Oil β-Triketones and Palmarosa Oil or Patchouli Oil

An ex vivo pig skin study was used to determine the effects of two test samples on relieving the impairment of the skin barrier that is characteristic of eczema, psoriasis, acne and other skin conditions, and cosmetic procedures where the skin may be pierced, treated with abrasive materials or otherwise damaged. The two extracts tested were: (a) manuka oil β-triketones and palmarosa oil and (b) manuka oil β-triketones and patchouli oil. The fresh skin from a pig (sourced from a butcher) was cut into pieces. For each test sample, three pieces of skin did not have the subcutaneous layer removed so as to serve as a control. Another three pieces had the subcutaneous layer removed using adhesive tape and then exposed to the test sample. A third set of three pieces had the subcutaneous layer removed using adhesive tape but were not exposed to the test samples and therefore served as a control for the effect of the test sample. Methylene blue-caffeine was added to the external surface of the skin pieces and the rate of the transfer through the skin pieces determined using Franz diffusion cells. Samples were taken from the underside of the cells at several intervals and used to determine the concentration of methylene blue. From each sample collected, 0.2 ml was dispensed into wells of a flat-bottomed 96-well plate. Using a VersaMax™ plate reader, the absorbance in each well was measured at 650 nm and recorded. The absorbance versus time was plotted graphically for each cell of the diffusion apparatus for each of the three transport conditions. The rate of transport of the methylene blue-caffeine across the skin was compared for each of the three conditions for both test samples.

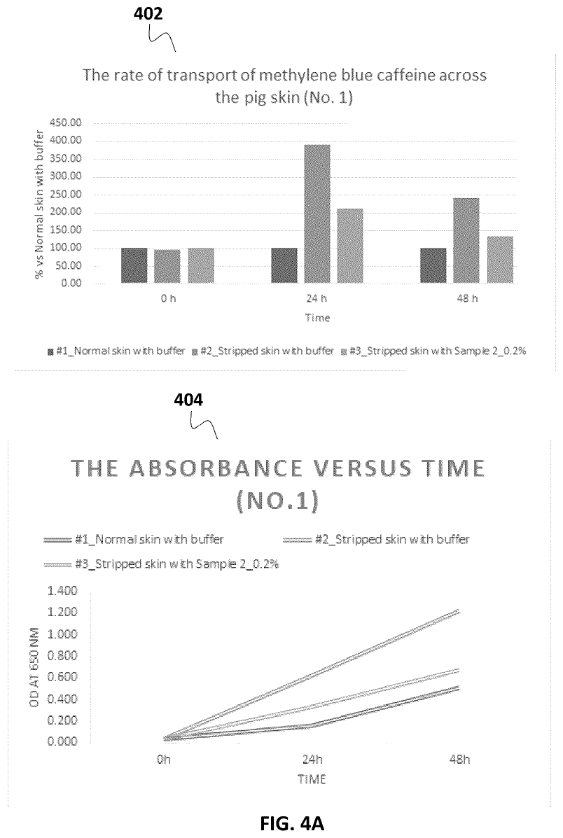

The results summarised in FIG. 4 demonstrate that, of the two samples tested, the extract with manuka oil β-triketones and palmarosa oil was more effective than the composition with manuka oil β-triketones and patchouli oil in restoring the skin barrier function in this study. FIG. 4A is graphs 402 and 404 showing skin barrier repair study results for 1 hour treatment of skin with 0.2% MBS β-triketones+0.2% palmarosa oil. FIG. 4B is graphs 406 and 408 showing 1 hour treatment of skin with 0.2% MBS β-triketones+0.2% patchouli oil.
(b) Treatment of skins with 0.2% MBS β-triketones+0.2% patchouli oil (1 hour)

### Example 16—Treatment of Psoriasis and Eczema

Three topical compositions are prepared including Leptospermum scoparium oil (manuka oil or β-triketones extracted from manuka oil) and i) CBD oil or isolate, ii) Cymbopogon martinii (palmarosa oil), (iii) patchouli oil, or iv) a combination of two or more of CBD, palmarosa, or patchouli oils as described above. The topical compositions are prepared and tested against skin ailments including psoriasis and eczema. The formulations demonstrate a therapeutic effectiveness in the treatment of one or more of the above skin ailments.

### Example 17—Topical Cream for the Treatment of Eczema, Psoriasis and Other Inflammatory Skin Conditions

A mixture of the components shown in Table 33 is prepared in the form of a topical cream for the treatment of eczema, psoriasis and other inflammatory skin conditions.

TABLE 33

| | Amount |
|---|---|
| Component | (wt. %) |
| Manuka oil (or β-triketones from manuka oil) | 0.5-10 |
| CBD oil, palmarosa oil, patchouli oil, or combinations | 0.1-10.0 |
| thereof | |
| PEG 300 | 30-60 |
| PEG 3350 | 10-20 |
| White soft paraffin | 5-20 |
| Stearyl alcohol | 5-20 |
| Propylene glycol | 0.5-10 |
| Sodium lauryl sulphate | <1% |
| Water | 0 to balance |

### Example 18—Treatment Against Skin Conditions

Topical compositions are prepared including Leptospermum scoparium oil (manuka oil or β-triketones extracted from manuka oil) and
a. CBD oil or isolate, or
b. Cymbopogon martinii (palmarosa oil), or
c. patchouli oil, or
d. a combination of two or more of CBD, palmarosa, or patchouli oils as described above and/or
e. one or more of the following:
   i. Nigella sativa seed oil (black seed oil);
   ii. Matricaria recutita oil (German chamomile);
   iii. Elettaria cardamomum oil (cardamom);
   iv. Thymus vulgaris oil (thyme);
   v. Origanum vulgare oil (oregano);
   vi. Santalum album oil (sandalwood);
   vii. Illicium verum oil (star anise);
   viii. Mentha spicata oil (spearmint)
   ix. Centella asiatica (gotukola extract)
   x. β-caryophyllene,
   xi. Psoralea corylifolia oil (babchi or bakuchi)
   xii. Terminalia ferdinandiana oil (Kakadu plum)

The topical compositions are prepared and tested against various skin ailments including skin aging, collagen production, scarring, UV damage, inflammation, itching, and yeast or fungi growth. The formulations demonstrate a therapeutic effectiveness in the treatment of one or more of the above skin ailments.

### Example 19—Treatment Against Viruses that Cause Cold Sores or Shingles

Three topical compositions are prepared including Leptospermum scoparium oil (manuka oil or β-triketones extracted from manuka oil) and i) CBD oil or isolate, ii) Cymbopogon martinii (palmarosa oil), (iii) patchouli oil, or iv) star anise oil, (v) α-bisabolol, vi) β-caryophyllene, (vii) thyme oil or manuka oil (or β-triketones extracted from manuka oil) and any combination of the oils described above. The topical compositions are prepared and tested against viruses such as, Human alpha herpes virus 1, Human alpha herpes zoster virus attributed to ailments such as cold sores or shingles. The formulations demonstrate a therapeutic effectiveness in the treatment of cold sores and shingles.

### Example 20—Demonstration of Purity and Consistency of Manuka Oil β-Triketones

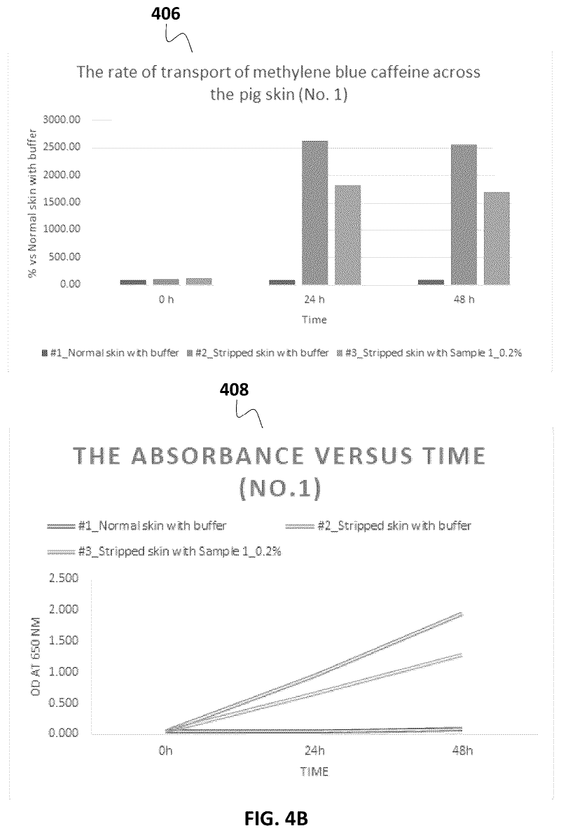

Active ingredients used in therapeutics must be pure and consistent. GCMS studies demonstrate that East Cape manuka oil β-triketones are pure and total level of triketones between batches is relatively consistent. FIG. 5 summarises the GCMS results for different batches of β-triketones. FIG. 5 is GCMS results 502 for manuka oil β-triketones with a table 504 summarizing the results. Table 504 summarizes GCMS results on several different batches of β-triketones. These results show that the total β-triketones are relatively stable across five batches, ranging from 761 to 781 mg/g. (Average value of 769±9 mg/g).

### Example 21—Demonstration of Chemical Inertness when Manuka β-Triketones and Palmarosa Oil are Combined

Much of the data included in this application demonstrates synergistic biological activity between manuka oil β-triketones and palmarosa oil.

In this context, it is important to establish that manuka β-triketones and palmarosa oil do not chemically react together since such reactivity can result in by-products that can cause sensitisation, allergenicity or other adverse reactions when used topically. The two ingredients were combined and the GCMS profile of the combination was compared with GCMS profiles of the individual ingredients.

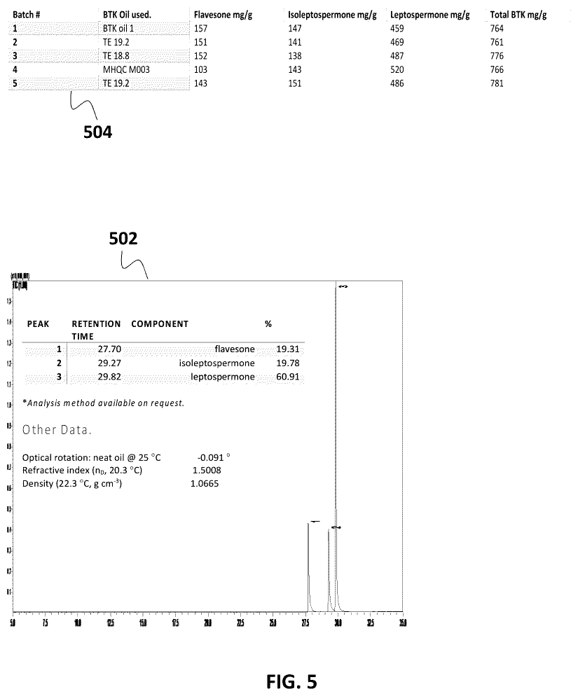

The results in FIG. 6 confirm that there is no chemical change when manuka β-triketones and palmarosa oil are combined. FIG. 6 is a graph 602 of GCMS results for manuka oil β-triketones, palmarosa oil and the combined composition.

### Example 22—Demonstration of Skin Compatibility of a Topical Composition Containing Manuka Oil b-Triketones and Palmarosa Oil in a Polyethylene Glycol Carrier Base

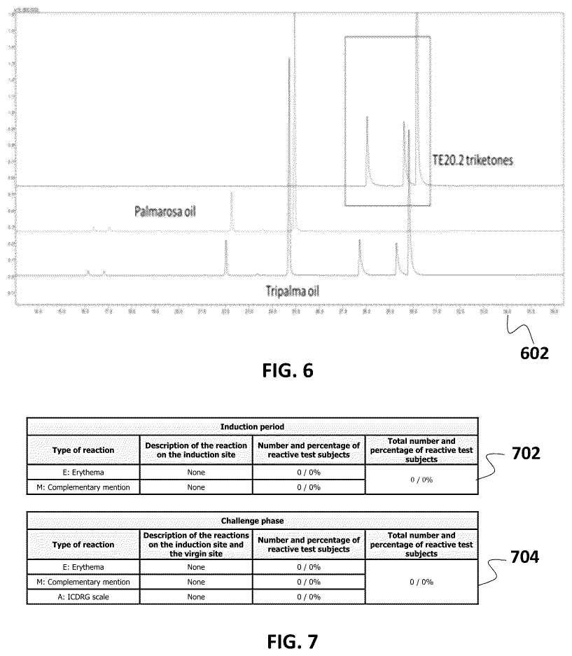

FIG. 7 summarises Human Repeat Insult Patch test (HRIPT) results for a topical composition comprising manuka oil β-triketones, palmarosa oil in a polyethylene glycol carrier base (named ECMT-154 PEG ointment, sometimes known as Tripalma). FIG. 7 is tables 702, 704 showing HRIPT results for a topical composition comprising manuka oil β-triketones, palmarosa oil in a polyethylene glycol carrier base (named ECMT-154 or Tripalma). Under the experimental conditions adopted (52 subjects with ages 19-68, phototype II-IV, with application to all types of skin) no reaction of irritation was induced during the induction phase, shown in graph 702. During the challenge phase, shown in graph 704, a single application of the product to the induction site and a virgin site induced no allergic reaction. Together, these results indicate very good skin compatibility and does not show a sensitizing effect.

### Example 23—MIC DATA (% v/v) for ECMO, ECMT, Palmarosa Oil & ECMT-154

TABLE 33 demonstrates the effects when 0.66% East Cape Manuka Triketones (ECMT) and 0.34% palmarosa oil are combined to produce ECMT-154, showing a clear synergistic effect against gram positive bacteria S. aureus, S. pyogenes, MRSA, S. epidermidis and C. acnes. The average MIC of palmarosa oil alone is 64 times that of ECMT-154. The average MIC of ECMT alone is 10.5 times that of ECMT-154.

If the antibacterial effect of the mixture was additive, an ECMT-154 MIC of 0.057% based on 0.66(0.021)+0.34(0.128)=0.057% would be expected. The observed average MIC of ECMT-154 against these gram positive bacteria (0.002%) is 1/29^{th} that of the expected additive figure. Furthermore, the observed average MIC of ECMT-154 against these bacteria (0.002%) is 1/32^{th} that of ECMO alone.

Overall, there is a minor synergistic effect against gram negative bacteria, E. coli, A. baumannii, and P. vulgaris. It may be possible to increase the gram-negative activity of ECMT-154 by increasing the concentration of palmarosa oil in the formulation. The average MIC of palmarosa oil alone is 0.6 times that of ECMT-154. The average MIC of ECMT alone is 4 times that of ECMT-154. If the antibacterial effect of the mixture was additive, we would expect an ECMT-154 MIC of 0.54%, based on 0.66(0.76)+0.34(0.113)=0.54%.

The observed average MIC of ECMT-154 against these gram-negative bacteria (0.193%) is ⅓^{rd} that of the expected additive figure. Furthermore, the observed average MIC of ECMT-154 against these bacteria (0.193%) is 1/9^{th} that of ECMO alone.

Overall, there is a minor synergistic effect against fungi and yeasts T. mentagrophytes, T. rubrum and C. albicans. It may be possible to increase the anti-fungal activity of ECMT-154 by increasing the concentration of palmarosa oil in the formulation. The average MIC of palmarosa oil alone is 1.2 times that of ECMT-154. The average MIC of ECMT alone is 4 times that of ECMT-154. If the antifungal effect of the mixture was additive, we would expect an ECMT-154 MIC of 0.24%, based on 0.66(0.313)+0.34(0.093)=0.24%. The observed average MIC of ECMT-154 against these fungi and yeasts (0.075%) is ⅓^{rd} that of the expected additive figure.

Furthermore, the observed average MIC of ECMT-154 against these fungi and yeasts (0.075%) is 1/7^{th} that of ECMO alone.

TABLE 33
Comparative MIC Data (% v/v): ECMO,
ECMT, Palmarosa Oil & ECMT-154

| | | | | |
|---|---|---|---|---|
| | | | | ECMT + |
| | ECMO | ECMT | Palmarosa | Palmarosa Oil |
| PATHOGEN | Only | Only | Oil only | ECMT-154 |

Gram positive bacteria

| | | | | |
|---|---|---|---|---|
| S aureus | 0.016 | 0.006 | 0.110 | 0.001 |
| S pyogenes | 0.125 | 0.063 | | 0.005 |
| MRSA | 0.040 | 0.007 | 0.200 | 0.003 |
| S. epidermidis | 0.064 | 0.010 | 0.1 | 0.001 |
| C. acnes | 0.070 | | 0.1 | 0.001 |
| AVERAGE | 0.063 | 0.021 | 0.128 | 0.002 |

Gram negative bacteria

| | | | | |
|---|---|---|---|---|
| E. coli | 2.100 | 1.200 | 0.125 | 0.165 |
| A. baumanni | | | 0.100 | 0.330 |
| P. vulgaris | 1.300 | 0.320 | | 0.083 |
| AVERAGE | 1.700 | 0.760 | 0.113 | 0.193 |

Fungi & yeast

| | | | | |
|---|---|---|---|---|
| T. mentagrophytes | 0.060 | 0.120 | 0.085 | 0.060 |
| T. rubrum | 0.170 | 0.320 | | 0.083 |
| C. albicans | 1.400 | 0.500 | 0.100 | 0.083 |
| AVERAGE | 0.543 | 0.313 | 0.093 | 0.075 |

### Example 24: MIC DATA (% v/v) for ECMO, ECMT, Patchouli Oil & ECMT-222

The data in TABLE 34 demonstrates the effects when 0.66% East Cape Manuka Triketones (ECMT) and 0.34% patchouli oil are combined to produce ECMT-222. There is a small synergistic effect against gram positive bacteria.

The average MIC of patchouli oil alone is 8 times that of ECMT-222. The average MIC of ECMT alone is 1.6 times that of ECMT-222. If the antibacterial effect of the mixture was additive, we would expect an ECMT-222 MIC of 0.051%, based on 0.66(0.021)+0.34(0.108)=0.051%. The observed average MIC of ECMT-222 against these gram-positive bacteria (0.0.013%) is ¼^{th} that of the expected additive figure. Furthermore, the observed average MIC of ECMT-222 against these bacteria (0.013%) is ⅕^{th} that of ECMO alone.

TABLE 34
Comparative MIC Data (% v/v): ECMO,
ECMT, Patchouli Oil & ECMT-222

| | | | | |
|---|---|---|---|---|
| | ECMO | ECMT | Patchouli | ECMT + Patchouli Oil |
| PATHOGEN | Only | Only | Oil Only | ECMT-222 |

Gram positive bacteria

| | | | | |
|---|---|---|---|---|
| S aureus | 0.016 | 0.006 | 0.250 | 0.005 |
| S pyogenes | 0.125 | 0.063 | | |
| MRSA | 0.040 | 0.007 | | |
| S. epidermidis | 0.064 | 0.010 | 0.025 | |
| C. acnes | 0.070 | | 0.050 | 0.021 |
| AVERAGE | 0.063 | 0.021 | 0.108 | 0.013 |

### Example 25: MIC DATA (% v/v) for ECMO, ECMT, CBD, ECMO-314 & ECMT-314

Cannabidiol (CBD) content in CBD oil can be highly variable. For this reason, pure CBD isolate was used in these examples. 1% ECMT or 1% ECMO were used in these examples. Since CBD oil contains 5-10% CBD, a concentration of 0.05% CBD pure isolate (equating to 0.5% to 1% whole CBD oil) was used in these tests.

TABLE 35
Comparative MIC Data (% v/v): ECMO,
ECMT, CBD Isolate & ECMT-314

| | | | | | |
|---|---|---|---|---|---|
| | | | | ECMO + | ECMT + |
| | ECMO | ECMT | CBD | CBD | CBD |
| PATHOGEN | Only | Only | Only | ECMO-314 | ECMT-314 |

Gram positive bacteria

| | | | | | |
|---|---|---|---|---|---|
| S aureus | 0.125 | 0.125 | 0.05 | 0.063 | 0.5 |
| MRSA | 0.125 | 0.125 | 0.0125 | 0.125 | 0.125 |
| S pyogenes | 0.063 | 0.031 | 0.002 | 0.063 | 0.031 |
| C. acnes | 0.125 | 0.25 | 0.006 | 0.125 | 0.0063 |
| AVERAGE | 0.110 | 0.133 | 0.018 | 0.094 | 0.166 |

The data in TABLE 35 demonstrates the effects when 1% East Cape Manuka oil or 1% Triketones (ECMT) and 0.05% pure CBD isolate are combined to produce ECMO-314 or ECMT-314. Overall, there appears to be a negligible additive effect against the tested gram-positive bacteria in the case of ECMO-314, and no additive effect against the tested gram positive bacteria in the case of ECMT-314. The average MIC of CBD alone is 0.2 times that of ECMO-314. The average MIC of CBD alone is 0.1 times that of ECMT-314. The average MIC of ECMO alone is 1.2 times that of ECMO-314. The average MIC of ECMT alone is 0.8 times that of ECMT-314.

If the gram-positive antibacterial effect of the mixture in ECMO-314 was additive, we would expect an ECMO-314 MIC of 0.1056% based on 0.9524(0.110)+0.0476(0.018)=0.1056. The observed average MIC of ECMO-314 against these gram-positive bacteria (0.094%) is 89% of the expected additive figure.

Similarly, if the antibacterial effect of the mixture in ECMT-314 was additive, we would expect an ECMT-314 MIC of 0.1275% based on 0.9524(0.133)+0.0476(0.018)=0.1275. The observed average MIC of ECMT-314 against these gram-positive bacteria (0.166%) is 130% of the expected additive figure.

If the C. acnes results for ECMT-314 are considered on its own, there is a strong synergistic result when ECMT and CBD are combined. This is included in [118] in the PCT application (page 43). Under this scenario, MIC of CBD alone is 0.95 times that of ECMT-314 and MIC of ECMT alone is 40 times that of ECMT-314.

If the anti-acne bacterial effect of the mixture in ECMT-314 was additive, we would expect an ECMT-314 MIC of 0.2384% based on 0.9524(0.25)+0.0476(0.006)=0.2384. The observed average MIC of ECMT-314 against C. acnes (0.0063%) is 1/38^{th} of the expected additive figure.

1. A topical composition for treatment of a skin condition comprising a mixture of:
Leptospermum scoparium oil (manuka oil), or β-triketones extracted from manuka oil;
at least one of Cymbopogon martinii (palmarosa oil), CBD oil, or Pogostemon cablin (patchouli oil); and
a carrier composition.

2. The topical composition of claim 1, wherein the topical composition comprises manuka oil having a base β-triketones content of at least 5 weight percent (wt. %).

3. The topical composition of claim 2, wherein the topical composition comprises manuka oil having a base β-triketones content of at least 20 wt. %.

4. The topical composition of claim 1, wherein the topical composition comprises about 0.05 weight percent (wt. %) to about 10 wt. % manuka oil or β-triketones extracted from manuka oil and about 0.05 wt. % to about 10 wt. % palmarosa oil based on a total weight of the topical composition.

5. The topical composition of claim 4, wherein the topical composition comprises about 0.05 weight percent (wt. %) to about 2 wt. % manuka oil or β-triketones extracted from manuka oil and about 0.05 wt. % to about 2 wt. % palmarosa oil based on a total weight of the topical composition.

6. The topical composition of claim 4, wherein the topical composition comprises about 0.05 weight percent (wt. %) to about 1 wt. % manuka oil or β-triketones extracted from manuka oil and about 0.05 wt. % to about 1 wt. % palmarosa oil based on a total weight of the topical composition.

7. The topical composition of claim 4, wherein the topical composition comprises greater than about 1 wt. % β-triketones provided from the manuka oil based on a total weight of the topical composition.

8. The topical composition of claim 4, wherein the topical composition comprises greater than about 2 wt. % β-triketones provided from the manuka oil based on a total weight of the topical composition.

9. (canceled)

10. The topical composition of claim 4, wherein the topical composition comprises greater than about 2 wt. % palmarosa oil.

11. The topical composition of claim 4, wherein the topical composition comprises about 1:3 ratio to about 3:1 ratio of palmarosa oil to β-triketones provided from the manuka oil.

12. The topical composition of claim 4, wherein the topical composition comprises about 1.5:1 to about 1:1.5 ratio of palmarosa oil to β-triketones provided from the manuka oil.

13. The topical composition of claim 4, wherein the carrier contains polyethylene glycol, allantoin, aloe, disodium EDTA, phenoxyethanol, decadiene cross polymer, triethylene glycol, stearyl alcohol, white soft paraffin, propylene glycol, lactic acid, glycerol and water.

14. The topical composition of claim 4, wherein the carrier comprises polyethylene glycol.

15. The topical composition of claim 4, further comprising CBD oil or isolate, Pogostemon cablin (patchouli oil); Nigella sativa seed oil (black seed oil), Matricaria recutita oil (German chamomile), Elettaria cardamomum oil (cardamom), Thymus vulgaris oil (thyme), Origanum vulgare oil (oregano), Santalum album oil (sandalwood), Illicium verum oil (star anise), Mentha spicata oil (spearmint), Centella asiatica (gotukola extract), β-caryophyllene, Psoralea corylifolia oil (babchi or bakuchi), or Terminalia ferdinandiana oil (Kakadu plum).

16. The topical composition of claim 4, wherein the topical composition is in the form of a topical balm, or a topical ointment, a topical cream, topical gel, topical solution, topical spray or topical oil.

17. The topical composition of claim 4, wherein the carrier composition comprises predominately water or an alcohol.

18. The topical composition of claim 4, wherein the carrier composition comprises one or more natural waxes, antioxidants, emollients, moisturizers, or combinations thereof.

19. The topical composition of claim 4, wherein the carrier composition comprises one or more of beeswax, Rhus succedanea fruit wax, Rhus verniciflua peel wax, Ricinus communis seed oil (castor oil), Simmondsia chinensis seed oil (jojoba seed oil), ascorbyl palmitate, and tocopherol.

20. The topical composition of claim 4, wherein the topical composition is for use in treating impetigo.

21. The topical composition of claim 4, wherein the topical composition is for use in treating psoriasis or eczema.

22. The topical composition of claim 4, wherein the topical composition is for use in treating or preventing bacterial skin or wound infections, fungal or yeast infections and herpes infections including cold sores and shingles.

23. A method for treating a skin condition comprising:
applying a topical composition to an area containing a skin condition, the topical composition comprising:
Leptospermum scoparium oil (manuka oil or β-triketones extracted from manuka oil);
at least one of CBD oil, Cymbopogon martinii (palmarosa oil), or Pogostemon cablin (patchouli oil); and
a carrier composition, wherein the manuka oil (or β-triketones extracted from manuka oil), and at least one of CBD oil, palmarosa oil, or patchouli oil are mixed in the carrier composition.

24. The method of claim 23, wherein the topical composition is in the form of a topical gel, topical ointment, topical cream, or topical balm, topical solution, topical spray or topical oil.

25. The method of claim 23, wherein the topical composition comprises palmarosa oil.

26. The method of claim 25, wherein the topical composition comprises about 1:2 ratio to about 2:1 ratio of palmarosa oil to β-triketones provided from the manuka oil.

27. The method of claim 26, further comprising mixing at least CBD oil or isolate, Pogostemon cablin (patchouli oil), Nigella sativa seed oil (black seed oil), Matricaria recutita oil (German chamomile), Elettaria cardamomum oil (cardamom), Thymus vulgaris oil (thyme), Origanum vulgare oil (oregano), Santalum album oil (sandalwood), Illicium verum oil (star anise), Mentha spicata oil (spearmint), Centella asiatica (gotukola extract), β-caryophyllene, Psoralea corylifolia oil (babchi or bakuchi), or Terminalia ferdinandiana oil (Kakadu plum) into the carrier composition.

28. The method of claim 26, wherein the carrier composition comprises polyethylene glycol.

29. A method of manufacturing a topical composition used to treat a skin condition comprising mixing Leptospermum scoparium oil (manuka oil or β-triketones extracted from manuka oil) and Cymbopogon martinii (palmarosa oil) into a carrier composition.

30. The method of claim 29, wherein the carrier composition comprises polyethylene glycol.

31. A topical composition for treatment of impetigo, the composition comprising:
a therapeutic mixture of:
Leptospermum scoparium oil (manuka oil or β-triketones extracted from manuka oil);
Cymbopogon martinii (palmarosa oil); and
a carrier composition.

32. The topical composition of claim 31, wherein the topical composition comprises about 1:2 ratio to about 2:1 ratio of palmarosa oil to β-triketones provided from the manuka oil.

33. The topical composition of claim 31, wherein the carrier composition comprises polyethylene glycol.
